(19) United States
(12) Patent Application Publication
(10) Pub. No.: US 2024/0299484 A1
(43) Pub. Date: Sep. 12, 2024

(54) DILEUCINE COMPOSITIONS AND METHODS OF USE THEREOF

(71) Applicant: Ingenious Ingredients, LP, Lewisville, TX (US)

(72) Inventors: Ralf Jäger, Whitefish Bay, WI (US); Martin Purpura, Spring, TX (US); Shawn D. Wells, Frisco, TX (US); Kylin Liao, Plano, TX (US)

(21) Appl. No.: 18/601,590

(22) Filed: Mar. 11, 2024

Related U.S. Application Data
(60) Provisional application No. 63/451,166, filed on Mar. 9, 2023.
(60) Provisional application No. 63/469,273, filed on May 26, 2023.

(51) Int. Cl. A61K 38/05; A61K 31/198; A61K 31/522; A61K 45/06; A61P 3/04; A61P 3/06; A61P 29/00; A61P 37/04; A61P 43/00

(52) U.S. Cl. CPC A61K 38/05; A61K 31/198; A61K 31/522; A61K 45/06; A61P 3/04; A61P 3/06; A61P 29/00; A61P 37/04; A61P 43/00

(57) ABSTRACT

Disclosed herein are compositions and methods for promoting weight loss and/or preventing or obesity; improving athletic performance/endurance; improving muscle function; improving cognitive function; promoting weight management; improving cardiovascular health; improving blood flow; reducing inflammation; and/or immune modulation. In certain aspects, the disclosed method comprises administering a composition to a subject comprising combinations of dileucine and paraxanthine and/or dileucine and L-arginine and/or dileucine and leucine.

### CROSS-REFERENCE TO RELATED APPLICATION(S)

This application claims priority to U.S. Provisional Application No. 63/451,166, filed Mar. 9, 2023, and entitled “DILEUCINE COMPOSITIONS AND METHODS OF USE THEREOF,” which is hereby incorporated by reference in its entirety under 35 U.S.C. § 119(e).

### BACKGROUND

Obesity is a significant problem in the Western world, with estimates of its prevalence ranging from 30% to 50% of the middle-aged population. The number of overweight (defined as a person with a body mass index (BMI) equal to or greater than 25 kg/m2) and obese (defined as a person with a BMI equal to or greater than 30 kg/m2) Americans has continued to increase since 1960, a trend that is not slowing down. Today, approximately 64.5% of adult Americans (about 199 million) are categorized as being overweight or obese. Obesity is becoming a growing concern as the number of people with obesity continues to increase and more is learned about the negative health effects of obesity. Obesity can lead to type 2 diabetes, heart disease, and some cancers. Each year, obesity causes at least 300,000 deaths in the U.S., and healthcare costs of American adults with obesity amount to more than $147 billion. There is a need in the art for compositions and methods to more effectively achieve weight loss.

### BRIEF SUMMARY

Disclosed herein is a composition comprising: paraxanthine and dileucine or L-arginine. According to certain embodiments, dileucine is present in an amount of from about 10 mg to about 5,000 mg. In further embodiments, dileucine is present in an amount of from about 25 mg to about 2,500 mg. In certain embodiments, paraxanthine is present in an amount of from about 2 mg to about 800 mg. In further embodiments, wherein paraxanthine is present in an amount from about 20 mg to about 600 mg. In yet further embodiments, paraxanthine is present in an amount from about 50 mg to about 400 mg. According to still further embodiments, L-arginine is present in an amount of about 500 mg to about 6,000 mg. In yet further embodiments, L-arginine is present in an amount of about 1,000 mg to about 3,000 mg. According to certain implementations, the L-arginine is in the form of a free form, salt, bonded arginine silicate complex, and/or arginine alpha-ketoglutarate.

Further disclosed herein is a method of improving muscle strength, muscle size, and/or muscle function comprising: administering to a subject a composition disclosed herein. In certain aspects of these embodiments, the composition further comprises one or more compounds selected from the list consisting of: isoleucine, leucine, and valine, histidine, lysine, methionine, phenylalanine, threonine, tryptophan, valine, creatine, cysteine, glutamine, glycine, proline, tyrosine, carnitine, beta-alanine, taurine, beta-hydroxy beta-methylbutyrate, Omega-3 fatty acids, Vitamin D, whey protein, and other protein extracts from animal, plant or fermentation sources.

Further disclosed herein is a method for increasing athletic performance in a subject by administering to the subject a composition disclosed herein.

Further disclosed herein is a method for increasing athletic performance and/or endurance in a subject by administering a composition disclosed herein.

Further disclosed herein is a method for promoting weight loss in a subject, administering a composition disclosed herein. In certain implementations, the composition further comprises one or more compounds selected from a list consisting of: caffeine, green tea, capsaicin, garcinia cambogia, yohimbine and bitter orange. In yet further implementations, weight loss is promoted through enhancing lipolysis in the subject and the composition further comprises one or more compounds selected from a list consisting of caffeine, green tea extract, L-carnitine, Garcinia cambogia (hydroxycitric acid), capsaicin, ginseng, taurine, silk peptides, catechols, epigallocatechin gallate (EGCG), catechins, proanthocyanidins and octacosanol.

Further disclosed herein is a method of improving cognitive performance in a subject by administering a composition disclosed herein. In certain aspects of these embodiments, improved cognitive function is measured by an increase in one or more of: attention, information acquisition, information processing, working memory, short-term memory, long-term memory, anterograde memory, retrograde memory, memory retrieval, discrimination learning, decision-making, inhibitory response control, attentional set-shifting, delayed reinforcement learning, reversal learning, the temporal integration of voluntary behavior, speed of processing, reasoning, problem solving and/or social cognition.

Further disclosed herein is a method of treating an inflammatory disease or condition in a subject in need thereof by administering a composition disclosed herein. In certain implementations of these embodiments, the composition further comprises a further anti-inflammatory agent wherein the further anti-inflammatory agent exerts anti-inflammatory effects by: inhibiting prostaglandins, increasing macrophage mediated phagocytosis, suppressing cytokine-driven inflammation, inhibiting cytokines, inhibiting histone deacetylation, inhibiting kinases, stimulating PPAR and/or inhibiting proteases.

Further disclosed herein is method for improving joint health in a subject in need thereof by administering a composition disclosed herein.

Further disclosed herein is method for enhancing immune function in a subject by administering a composition disclosed herein. In certain implementations of these embodiments, the composition further comprises one or more compounds selected from the list consisting of: Cyclosporine, tacrolimus, rapamycin, Omega-3 fatty acids, Curcumin, S-adenosylmethionine, Zinc, Green tea extract, Frankincense (Boswellia serrata resin), Capsaicin, Cat's claw (uncaria plants, including Uncaria tomentosa and Uncaria guianensis), Schizonepeta tenuifolia, Pomegranate, Moringa oleifera, Ecklonia cava, Limonene, Sunifiram, Sulforaphane, Angelica gigas, Ascophyllum nodosum, Scutellaria baicalensis, celery seed extract, Sesamin, Feverfew, Taurine, Rosmarinic Acid, Evodia rutaecarpa, Green Tea Catechins, Punicalagins, Artemisia iwayomogi, Pyrroloquinoline quinone, N-Acetylcysteine, King Oyster, Methylsulfonylmethane, alpha-lipoic acid, pine pollen, Sophora flavescens, Ophiopogon japonicus, Stephania tetrandra, Crataegus pinnatifida, grape seed extract, Bladderwrack, Pacderia foctida, Benfotiamine, Rubus coreanus, Punicic Acid, Sea Buckthorn, Hibiscus rosasinensis, Phellodendron amurense, Resveratrol, Quercetin, Rooibos, Olive leaf extract, Pterostilbene, Eucommia ulmoides, Diindolylmethane, Anatabine, Serrapeptase, Pelargonidin, watercress, Astaxanthin, Piccatannol, Fish Oil, Glutathione, Orthosiphon stamineus, Aronia melanocarpa, blueberry, Tripterygium wilfordii, Boerhaavia diffusa, Whey Protein, Bromelain, Panax ginseng, Aloe vera, cocoa extract, stinging nettle, garlic, Centella asiatica, Astragalus membranaceus, Dendrobium, Vitamin C, Spirulina, Berberine, Ganoderma lucidum, Vitamin C, Vitamin D, Vitamin E, lutein, leucine, dileucine, trileucine, tetraleucine Pau d'arco, AHCC, Rhodiola ashwagandha, shiake, maitake, turkey tail, monolaurin, lysine, Ergothioneine, medium chain triglycerides (MCTs) and butyrate.

Further disclosed herein is a method for improving cardiovascular health in a subject by administering a composition disclosed herein.

Further disclosed herein is a method for improving blood flow in a subject, by administering a composition disclosed herein.

While multiple embodiments are disclosed, still other embodiments of the disclosure will become apparent to those skilled in the art from the following detailed description, which shows and describes illustrative embodiments of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### DETAILED DESCRIPTION

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “subject” refers to the target of administration, e.g. a subject. Thus the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more body weight disorders, inflammatory disorders, cardiovascular disorders, and/or cognitive disfunction, prior to the administering step. In some aspects of the disclosed method, the subject has been diagnosed with a need for increasing weight loss prior to the administering step.

The term “overweight” is defined as the condition wherein the individual has a BMI greater than or 25 kg/m2 and less than 30 kg/m2. The terms “overweight” and “pre-obese” are used interchangeably.

As used herein, the term “obesity” is defined as the condition wherein the individual has a BMI equal to or greater than 30 kg/m2. According to a WHO definition the term obesity may be categorized as follows: the term “class I obesity” is the condition wherein the BMI is equal to or greater than 30 kg/m2 but lower than 35 kg/m2; the term “class II obesity” is the condition wherein the BMI is equal to or greater than 35 kg/m2 but lower than 40 kg/m2; the term “class III obesity” is the condition wherein the BMI is equal to or greater than 40 kg/m2.

As used herein, the terms “manage,” “managing,” and “management” encompass preventing, delaying, or reducing the severity of a recurrence of an adipose associated body composition or body weight disorder, such as obesity, lypodystrophy, diabetes or metabolic syndrome, fibrosis and cancer in a patient who has already suffered from such a disease, disorder or condition. The terms encompass modulating the threshold, development, and/or duration of the adipose associated body composition or body weight disorder, such as obesity, lypodystrophy, diabetes or metabolic syndrome, fibrosis and cancer or changing how a patient responds to the adipose associated body composition or body weight disorder.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinca pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with obesity” means having been subjected to a physical examination by a person of skill, for example, a physician, and found to have a condition that can be diagnosed or treated by a compound or composition that can reduce body mass. As a further example, “diagnosed with a need for weight loss” refers to having been subjected to a physical examination by a person of skill, for example, a physician, and found to have a condition characterized by excess of body fat or other disease wherein decreasing body fat would be beneficial to the subject. Such a diagnosis can be in reference to a disorder, such as obesity, metabolic syndrome, and the like, as discussed herein.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to excess adipose tissue) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a composition to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “cardiovascular disease” or “cardiovascular disorder” is used herein in the broadest sense and includes all diseases and pathological conditions the pathogenesis of which involves abnormalities of the blood vessels, such as, for example, atherosclerotic plaque formation (including stable or unstable/vulnerable plaques), atherosclerosis, arteriosclerosis, arteriolosclerosis, and elevated systemic lipopolysaccharide (LPS) exposure. The term additionally includes diseases and pathological conditions that benefit from the inhibition of the formation of atherosclerotic plaques. Cardiovascular diseases include, without limitation, coronary artery atherosclerosis, coronary microvascular disease, stroke, carotid artery disease, peripheral arterial disease, ischemia, coronary artery disease (CAD), acute coronary syndrome (ACS), coronary heart disease (CHD), conditions associated with CAD and CHD, cerebrovascular disease, peripheral vascular disease, aneurysm, vasculitis, venous thrombosis, diabetes mellitus, and metabolic syndromechronic kidney disease, remote tissue injury after ischemia and reperfusion, cardiopulmonary bypass. Specifically included within this group are all cardiovascular diseases associated with the occurrence, development, or progression of which can be controlled by the inhibition of the atherosclerotic plaque formation.

As used herein, “improving cardiovascular health” includes preventing, treating, and maintaining protection against hypercholesterolemia and hypertension and achieving and maintaining normal ratios of LDL, HDL and triglycerides

As used herein “inflammatory disease or condition” includes, but is not limited to: which the compositions and methods herein may be used include, but are not limited to: acquired immune deficiency syndrome (AIDS), acute disseminated encephalomyelitis (ADEM), Addison's disease, agammaglobulinemia, allergic diseases, alopecia areata, Alzheimer's disease, amyotrophic lateral sclerosis, ankylosing spondylitis, antiphospholipid syndrome, antisynthetase syndrome, arterial plaque disorder, asthma, atherosclerosis, atopic allergy, atopic dermatitis, autoimmune aplastic anemia, autoimmune cardiomyopathy, autoimmune enteropathy, autoimmune hemolytic anemia, autoimmune hepatitis, autoimmune hypothyroidism, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenia purpura, autoimmune urticarial, autoimmune uveitis, Balo disease/Balo concentric sclerosis, Behcet's disease, Berger's disease, Bickerstaffs encephalitis, Blau syndrome, bullous pemphigoid, Castleman's disease, celiac disease, Chagas disease, chronic inflammatory demyelinating polyneuropathy, chronic recurrent multifocal osteomyelitis, chronic obstructive pulmonary disease, chronic venous stasis ulcers, Churg-Strauss syndrome, cicatricial pemphigoid, Cogan syndrome, cold agglutinin disease, complement component 2 deficiency, contact dermatitis, cranial arteritis, CREST syndrome, Crohn's disease, Cushing's Syndrome, cutaneous leukocytoclastic angiitis, Dego's disease, Dercum's disease, dermatitis herpetiformis, dermatomyositis, Diabetes mellitus type I, Diabetes mellitus type II diffuse cutaneous systemic sclerosis, Dressler's syndrome, drug-induced lupus, discoid lupus crythematosus, eczema, emphysema, endometriosis, enthesitis-related arthritis, eosinophilic fasciitis, cosinophilic gastroenteritis, cosinophilic pneumonia, epidermolysis bullosa acquisita, erythema nodosum, erythroblastosis fetalis, essential mixed cryoglobulinemia, Evan's syndrome, fibrodysplasia ossificans progressive, fibrosing alveolitis (or idiopathic pulmonary fibrosis), gastritis, gastrointestinal pemphigoid, Gaucher's disease, glomerulonephritis, Goodpasture's syndrome, Graves' disease, Guillain-Barre syndrome (GBS), Hashimoto's encephalopathy, Hashimoto's thyroiditis, heart disease, Henoch-Schonlein purpura, herpes gestationis (aka gestational pemphigoid), hidradenitis suppurativa, HIV infection, Hughes-Stovin syndrome, hypogammaglobulinemia, infectious diseases (including bacterial infectious diseases), idiopathic inflammatory demyelinating diseases, idiopathic pulmonary fibrosis, idiopathic thrombocytopeni purpura, IgA nephropathy, inclusion body myositis, inflammatory arthritis, inflammatory bowel disease, inflammatory dementia, interstitial cystitis, interstitial pneumonitis, juvenile idiopathic arthritis (aka juvenile rheumatoid arthritis), Kawasaki's disease, Lambert-Eaton myasthenic syndrome, leukocytoclastic vasculitis, lichen planus, lichen sclerosus, linear IgA disease (LAD), lupoid hepatitis (aka autoimmune hepatitis), lupus erythematosus, lymphomatoid granulomatosis, Majeed syndrome, malignancies including cancers (e.g., sarcoma, Kaposi's sarcoma, lymphoma, leukemia, carcinoma and melanoma), Meniere's disease, microscopic polyangiitis, Miller-Fisher syndrome, mixed connective tissue disease, morphea, Mucha-Habermann disease (aka Pityriasis lichenoides et varioliformis acuta), multiple sclerosis, myasthenia gravis, myositis, narcolepsy, neuromyelitis optica (aka Devic's disease), neuromyotonia, occular cicatricial pemphigoid, opsoclonus myoclonus syndrome, Ord's thyroiditis, palindromic rheumatism, PANDAS (pediatric autoimmune neuropsychiatric disorders associated with streptococcus), paraneoplastic cerebellar degeneration, Parkinsonian disorders, paroxysmal nocturnal hemoglobinuria (PNH), Parry Romberg syndrome, Parsonage-Turner syndrome, pars planitis, pemphigus vulgaris, peripheral artery disease, pernicious anaemia, perivenous encephalomyelitis, POEMS syndrome, polyarteritis nodosa, polymyalgia rheumatic, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restenosis, restless leg syndrome, retroperitoneal fibrosis, rheumatoid arthritis, rheumatic fever, sarcoidosis, schizophrenia, Schmidt syndrome, Schnitzler syndrome, scleritis, scleroderma, sepsis, Sickness, serum Sjogren's syndrome, spondyloarthropathy, Still's disease (adult onset), stiff person syndrome, stroke, subacute bacterial endocarditis (SBE), Susac's syndrome, Sweet's syndrome, Sydenham chorea, sympathetic ophthalmia, systemic lupus erythematosus, Takayasu's arteritis, temporal arteritis (aka “giant cell arteritis”), thrombocytopenia, Tolosa-Hunt syndrome) transplant (e.g., heart/lung transplants) rejection reactions, transverse myelitis, tuberculosis, ulcerative colitis, undifferentiated connective tissue disease, undifferentiated spondyloarthropathy, urticarial vasculitis, vasculitis, vitiligo, and Wegener's granulomatosis.

The terms “modulate”, “modulating”, “modulation”, “enhance”, “enhancing”, and “enhancement” are used synonymously herein to describe the improved ability of any human or animal (including, but not limited to, a dog, cat, rodent, horse, sheep, cow, pig, goat, donkey, chicken, or rabbit) to mount an immune response.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “synergistic effect” or grammatical variations thereof means and includes a cooperative action encountered in a combination of two or more active compounds in which the combined activity of the two or more active compounds exceeds the sum of the activity of each active compound alone. The term “synergistically effective amount,” as used herein, means and includes an amount of two or more active compounds that provides a synergistic effect defined above.

Disclosed herein are compositions and methods for promoting weight loss and/or preventing or obesity; improving athletic performance/endurance; improving muscle function; improving cognitive function; promoting weight management; improving cardiovascular health; improving blood flow; reducing inflammation; and/or immune modulation. In certain aspects, the disclosed method comprises administering a composition to a subject comprising combinations of dileucine and paraxanthine and/or dileucine and L-arginine and/or dileucine and leucine.

Di-leucine refers to a dipeptide comprised of two L-leucines. In certain aspects, di-leucine can have the structure:

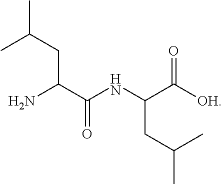 [SMILES: CC(C)CC(N)C(=O)NC(CC(C)C)C(=O)O]

Di-leucine may also be referred to as L-Leucyl-L-leucine or Leu-Leu and has number CAS #3303-31-9.

In certain aspects, disclosed are compositions comprising dileucine, leucine, and a pharmaceutically acceptable carrier thereof. In various aspects dileucine is present from about 10% to 90% (w/w). In further aspects, the dileucine is present from about 20% to 80% (w/w). In further aspects, wherein dileucine is present from about 30% to 70% (w/w). In further aspects, dileucine is present from about 40% to 60% (w/w). In still further aspects, dileucine is present from about 50% (w/w).

In alternative embodiments, dileucine is present from about 10%-90% (w/w) and leucine is present from about 90%-10% (w/w). In further embodiments, dileucine is present from about 20%-80% (w/w) and leucine is present from about 80%-20% (w/w). In further embodiments, dileucine is present from about 30%-70% (w/w) and leucine is present from about 70%-30% (w/w). In further embodiments, dileucine is present from about 40%-60% (w/w) and leucine is present from about 60%-40% (w/w). In further embodiments, dileucine is present at about 50% (w/w) and leucine is present at about 50% (w/w).

In certain aspects, the disclosed composition comprises di-leucine salt. In exemplary embodiments, the composition is a di-leucine acetate salt, which may have the structure:

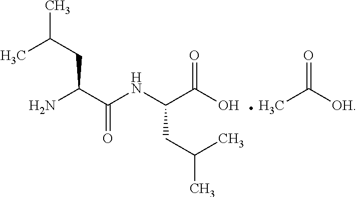

In certain aspects, the disclosed composition comprises tri-leucine. Trileucine means a tripeptide comprising of three L-leucines. In certain aspects, tri-leucine has the structure:

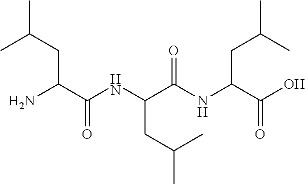 [SMILES: CC(C)CC(N)C(=O)NC(CC(C)C)C(=O)NC(CC(C)C)C(=O)O]

Tri-leucine may also be referred to as TRILEUCINE;LEU-LEU-LEU;H-LEU-LEU-LEU-OH;L-LEUCYLLEUCYLLEUCINE;leucyl-leucyl-leucine;Leu-leu-leucrystalline;L-LEUCY-L-LEUCYL-L-LEUCINE;L-LEUCYL-L-LEUCYL-L-LEUCINE;Leu-Leu-Leu-OH≥S)-2-((S)-2-((S)-2-Amino-4-methylpentanamido)-4-methylpentanamido)-4-methylpentanoic acid.

In further aspects, the disclosed method comprises administering a composition to a subject where the composition comprises tripeptide comprising two L-Leucine units and one amino acid or amino acid derivative. According to certain embodiments, the amino acid is selected from a group of branched-chain amino acids (BCAA), including, but not limited to, isoleucine, leucine, and valine. In further embodiments, the amino acid is selected from the group of essential amino acids, including, but not limited to, histidine, isoleucine, leucine, lysine, methionine, phenylalanine, threonine, tryptophan, and valine. In still further embodiments, the amino acid is selected from the group of conditionally essential amino acids including, but not limited to, arginine, cysteine, glutamine, glycine, proline, and tyrosine. According to certain embodiments, the conditionally essential amino acid is tyrosine. In exemplary embodiments, the composition comprises a di-leucine tyrosine that has the structure:

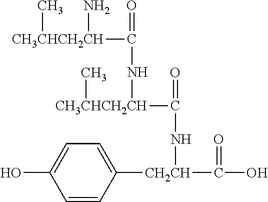

In still further embodiments, the amino acid is selected from the group of non-essential amino acids including, but not limited to, alanine, aspartic acid, asparagine, glutamic acid, serine, selenocysteine and pyrrolsine. In yet further embodiments, the amino acid derivative is selected from the group of creatine, carnitine, creatinol, beta-alanine, taurine, and beta-hydroxy beta-methylbutyrate.

In certain aspects, disclosed is a method for reducing body weight and/or body fat in a subject comprising administering to the subject an effective amount of a composition that comprises at least one amino acid or peptide chosen from: di-leucine, tri-leucine, and Leu-Leu-R, wherein R-is an amino acid or an amino acid derivative, and pharmaceutically acceptable salts thereof. In various aspects the at least one amino acid or peptide comprises leucine and di-leucine and/or and pharmaceutically acceptable salts thereof.

In certain embodiments, dileucine is present from about 10% (w/w) to about 90% (w/w). In further embodiments, dileucine is present from about 30% to 70% (w/w). In further embodiments, dileucine is present at about 50% (w/w).

In various alternative embodiments, dileucine is present from about 10%-90% (w/w) and leucine is present from about 90%-10% (w/w) (of the combined weight of leucine and dileucine). In further embodiments, dileucine is present from about 30%-70% (w/w) and leucine is present from about 70%-30% (w/w). In further embodiments, dileucine is present at about 50% (w/w) and leucine is present at about 50% (w/w). In each of the foregoing, % (w/w) refer to percentage relative to the combined weight of leucine and dileucine.

In these and other embodiments, the administration of the composition to the subject synergistically increases the plasma levels of leucine relative to administration of a composition comprising leucine without dileucine. Additionally, the administration of the composition to the subject synergistically reducing body weight and/or body fat relative to administration of a composition comprising leucine without dileucine.

In various aspects the compositions comprise at least about 95% dileucine; and between about 0.1%-5% tri-leucine, and pharmaceutically acceptable salts thereof. In further aspects, the tri-leucine is present at an amount between about 0.1%-3% and the composition further comprising about 0.1%-2% tetra-leucine. In yet further aspects, the tri-leucine is present at an amount of about 0.4% and the tetra-leucine is present at an amount of about 0.2%.

According to certain alternative embodiments, the composition comprises at least about 95% dileucine; and between about 0.1%-5% tetra-leucine, and pharmaceutically acceptable salts thereof.

In certain aspects of the foregoing embodiments, the composition is substantially free of leucine.

In certain aspects, the dileucine of the disclosed compositions are produced through bacterial fermentation. According to these embodiments, fermentation techniques are employed utilizing di-/tri-/tetra-peptide-forming enzymes that directly links amino acids, followed by extraction processes.

Paraxanthine, or 1,7-dimethylxanthine, is a dimethyl derivative of xanthine. Paraxanthine has the structure:

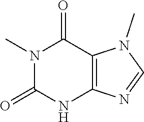

Paraxanthine in the disclosed compositions may be derived from natural sources, synthetic, and/or through fermentation.

In certain embodiments, the composition is formulated such that a dose paraxanthine is provided ranging from about 1 to about 1000 mg (e.g., about 1 mg, about 5 mg, about 10 mg, about 15 mg, about 20 mg, about 25 mg, about 30 mg, about 35 mg, about 40 mg, about 45 mg, about 50 mg, about 75 mg, 100, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg, and the like, or any range or value therein).

According to certain embodiments, the disclosed composition comprises paraxanthine and dileucine. In certain embodiments, paraxanthine is present in an amount of from about 2 mg to about 800 mg. In further embodiments, paraxanthine is present in amount from about 20 mg to about 600 mg. In yet further embodiments, paraxanthine is present in amount from about 50 mg to about 400 mg. In certain implementations, dileucine is present in an amount of from about 10 mg to about 5,000 mg. In further embodiments, dileucine is present in an amount of from about 25 mg to about 2,500 mg.

In certain embodiments, the disclosed composition comprises dileucine and L-arginine. Arginine is an amino acid with the formula (H2N)(HN)CN(H)(CH2)3CH(NH2)CO2H. At physiological pH, the carboxylic acid is deprotonated (˜CO2-) and both the amino and guanidino groups are protonated, resulting in a cation. Only the L-arginine (symbol Arg or R) enantiomer is found naturally. Arginine is classified as a semi-essential or conditionally essential amino acid, depending on the developmental stage and health status of the individual. Arginine in the instantly disclosed composition may be in a free form, salt, bonded arginine silicate complex, and/or arginine alpha-ketoglutarate and other sources of arginine known in the art.

In certain embodiments, dileucine is present in an amount of from about 10 mg to about 5,000 mg. In further embodiments, dileucine is present in an amount of from about 25 mg to about 2,500 mg. In certain implementations of the foregoing embodiments, L-arginine is present in an amount of about 500 mg to about 6,000 mg. In further embodiments, L-arginine is present in an amount of about 1,000 mg to about 3,000 mg. In certain embodiments, the composition comprises dileucine, paraxanthine, and L-arginine.

### Nutritional Supplements

The compositions of the disclosure may take the form of dietary supplements or may themselves be used in combination with dietary supplements, also referred to herein as food supplements.

Nutritional supplements may be found in many forms such as tablets, capsules, soft gels, gel caps, liquids, or powders. Some dietary supplements can help ensure an adequate dietary intake of essential nutrients; others may help reduce risk of disease.

### Food Products

The compositions of the disclosure may take the form of a food product. Here, the term “food” is used in a broad sense and covers food and drink for humans as well as food and drink for animals (i.e. a feed). Preferably, the food product is suitable for, and designed for, human consumption.

The food may be in the form of a liquid, solid or suspension, depending on the use and/or the mode of application and/or the mode of administration.

When in the form of a food product, the composition may comprise or be used in conjunction with one or more of: a nutritionally acceptable carrier, a nutritionally acceptable diluent, a nutritionally acceptable excipient, a nutritionally acceptable adjuvant, a nutritionally active ingredient.

By way of example, the compositions of the disclosure may take the form of one of the following: A fruit juice; a beverage comprising whey protein: a health or herbal tea, a cocoa drink, a coffee drink, a yoghurt and/or a drinking yoghurt, a cheese, an ice cream, a desserts, a confectionery, a biscuit, a cake, cake mix or cake filling, a snack food, a fruit filling, a cake or doughnut icing, an instant bakery filling cream, a filling for cookies, a ready-to-use bakery filling, a reduced calorie filling, an adult nutritional beverage, an acidified soy/juice beverage, a nutritional or health bar, a beverage powder, a calcium fortified soy milk, or a calcium fortified coffee beverage.

### Food Ingredients

Compositions of the present disclosure may take the form of a food ingredient and/or feed ingredient.

As used herein the term “food ingredient” or “feed ingredient” includes a composition which is or can be added to functional foods or foodstuffs as a nutritional and/or health supplement for humans and animals.

The food ingredient may be in the form of a liquid, suspension or solid, depending on the use and/or the mode of application and/or the mode of administration.

### Functional Foods

Compositions of the disclosure may take the form of functional foods.

As used herein, the term “functional food” means food which is capable of providing not only a nutritional effect but is also capable of delivering a further beneficial effect to the consumer.

Accordingly, functional foods are ordinary foods that have components or ingredients (such as those described herein) incorporated into them that impart to the food a specific function—e.g. medical or physiological benefit—other than a purely nutritional effect.

Although there is no legal definition of a functional food, most of the parties with an interest in this area agree that they are foods marketed as having specific health effects beyond basic nutritional effects.

Some functional foods are nutraceuticals. Here, the term “nutraceutical” means a food which is capable of providing not only a nutritional effect and/or a taste satisfaction, but is also capable of delivering a therapeutic (or other beneficial) effect to the consumer. Nutraceuticals cross the traditional dividing lines between foods and medicine.

### Medical Foods

Compositions of the present disclosure may take the form of medical foods.

By “medical food” it is meant a food which is formulated to be consumed or administered with or without the supervision of a physician and which is intended for a specific dietary management or condition for which distinctive nutritional requirements, based on recognized scientific principles, are established by medical evaluation.

Methods of Use and/or Treatment

In certain embodiments, dileucine may be combined with one or more other chemical compounds (e.g. other active ingredients), to provide a plurality of positive effects in a subject. By altering the dosage of dileucine and/or chemical compounds it is combined with, various physiological effects may be selected for. The compositions may provide primarily a single benefit or may provide multiple benefits simultaneously.

### Athletic Performance

Further disclosed herein is a method for enhancing performance or energy in subject, comprising administering to the subject a composition disclosed herein. As used herein the term “enhancing performance” is intended to mean any improvement in performance. Performance can be assessed in any manner. Certain enhancements are readily measured. For example, in a timed-event, an improved time can assess an enhanced performance. Certain performance enhancing properties can be judged subjectively by the athlete or performer or an observer. In these instances, an enhanced performance means that the performance was perceived subjectively to be improved, magnified, faster, better and the like. In certain embodiments, the disclosed methods are used to enhance athletic performance. “Athletic performance” refers to any professional or recreational activity wherein the performer, for example an athlete, exerts a physical act, such as running, swimming, golf, bowling, archery, football, baseball, basketball, soccer, hiking, cycling, dancing and the like. In certain athletic performance is improved through in improvement of endurance in the subject. In other words, administration of the disclosed compositions improves a subject's level of endurance, thereby enhancing the subject's athletic performance. In further embodiments, administration of the composition to the subject increases cognitive performance which thereby improves athletic performance.

In certain embodiments, upon administration of the composition, the subject experiences improvement of at least one of mood, energy, focus, concentration or sexual desire or a reduction of at least one of anxiety, fatigue, perception of effort or perception of pain.

In further embodiments, upon continued administration to the subject, the composition does not create dependence in the subject and/or withdrawal effect in the subject when continued use is ceased.

Further disclosed herein is a method of increasing athletic endurance in a subject comprising administering to the subject a composition disclosed herein. In certain implementations, the composition administered to the subject comprises dileucine and paraxanthine. In exemplary implementations, the administration of paraxanthine and dileucine produce a synergistic increase athletic endurance in the subject, relative to the administration of paraxanthine or dileucine alone.

According to further embodiments, administration of the disclosed composition to the subject increases the subject's perceived level of energy. In exemplary implementations, the subject experiences an increase in energy of at least about 5 percent. According to certain embodiments, the composition administered further comprises (in addition to dileucine and/or paraxanthine) at least one ingredient selected from the group consisting of L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, tetrahydrocurcumin, and Solanum asperum and/or combinations thereof, caffeine, theobromine, naringin, hesperidin, 2-(dimethylamino)ethanol (DMAE), DMAE bitartrate, huperzine A, theacrine, methylliberine, B12, sulbutiamine, magnolia bark, ketones, MCTs, omega 3's, lutein, zeaxanthin, tyrosine and n-acetyl-tyrosine, taurine, acetyl-1-carnitine and/or combinations thereof.

In certain aspects of the foregoing embodiments wherein the composition comprises effective amounts of dileucine and paraxanthine, the administration of paraxanthine and dileucine produce a synergistic improvement in athletic performance in the subject, relative to the administration of paraxanthine or dileucine alone.

In certain further aspects of the foregoing embodiments wherein the composition comprises effective amounts of dileucine and L-arginine, the administration of L-arginine and dileucine produce a synergistic increase in athletic performance in the subject, relative to the administration of L-arginine or dileucine alone.

### Muscle Function

Further disclosed herein is a method for increasing muscle function in a subject by administering to the subject a composition disclosed herein. In certain aspects, disclosed herein are methods to promote muscle growth through the administration of an effective amount of one or more compositions disclosed herein. In certain further aspects, administration of effective amounts of the disclosed compositions results in greater level of muscle protein synthesis (MPS) in the subject. In still further aspects, administration of effective amounts of the disclosed compositions results in improved muscle accretion in the subject.

In certain aspects, disclosed herein are methods to promote muscle growth through the administration of an effective amount of one or more compositions disclosed herein. In certain further aspects, administration of effective amounts of the disclosed compositions results in greater level of muscle protein synthesis (MPS) in the subject. In still further aspects, administration of effective amounts of the disclosed compositions results in improved muscle accretion in the subject.

According to certain embodiments, compositions disclosed herein may be administered in conjunction with a strength training regime. As will be appreciated by a person having skill in the art, administration of effective amounts of the disclosed compositions results in improved strength and improved athletic performance/ergogenesis in the subject.

In one aspect, the disclosed compounds inhibit muscle atrophy. In a further aspect, the disclosed compounds increase muscle mass. In a still further aspect, the disclosed compounds induce muscle hypertrophy. In a yet further aspect, the disclosed compounds inhibit of muscle atrophy and increase muscle mass. In an even further aspect, the disclosed compounds inhibit of muscle atrophy and induce muscle hypertrophy. In a further aspect, the inhibition of muscle atrophy is in a subject. In an even further aspect, the increase in muscle mass is in a subject. In a still further aspect, the subject is a mammal. In a yet further aspect, the mammal is a human.

In certain aspects, administration of the disclosed compositions is effective at preventing or treating age-related muscle atrophy or sarcopenia. In further aspects, administration of the disclosed compositions is effective at preventing or treating muscle atrophy associated with muscle immobilization, such as that which frequently occurs with casting of fractured bones. In further aspects, administration of the disclosed compositions is effective at preventing or treating muscle atrophy associated with disease, such as cancer, also known as cachexia.

According to certain aspects the composition is administered to a subject that has sarcopenia. In various aspects, the composition is administered in a therapeutically effective amount. In further aspects, the composition is administered at prophylactically effective amount, (e.g. to a subject at risk for developing sarcopenia, cachexia, or immobilization induced atrophy).

In certain aspects, the composition further comprises one or more additional active ingredient to further enhance muscle strength, size, and/or muscle function. In certain embodiments, the one or more additional active ingredient is an amino acid. According to certain embodiments, the amino acid is selected from a group of branched-chain amino acids (BCAA), including, but not limited to, isoleucine, leucine, and valine. In further embodiments, the amino acid is selected from the group of essential amino acids, including, but not limited to, histidine, isoleucine, leucine, lysine, methionine, phenylalanine, threonine, tryptophan, and valine. In still further embodiments, the amino acid is selected from the group of conditionally essential amino acids including, but not limited to, arginine, cysteine, glutamine, glycine, proline, ergothioneine, and tyrosine. According to the certain embodiments, the conditionally essential amino acid is tyrosine. In still further embodiments, the amino acid is selected from the group of non-essential amino acids including, but not limited to, alanine, aspartic acid, asparagine, glutamic acid, serine, selenocysteine and pyrrolysine. In yet further embodiments, the amino acid derivative is selected from the group of creatine, carnitine, beta-alanine, taurine, beta-hydroxy beta-methylbutyrate L-Arginine, omega-3 fatty acids, Vitamin D, whey protein, BAIBA, and other protein extracts from animal, plant or fermentation sources.

According to exemplary aspects of these embodiments, that may reduce fatigue, improve energy, increase mobility, and improve alertness. In further embodiments, administration of the disclose compositions is cardio protective. In further embodiments, administration of the disclose compositions improves muscle contractions and muscle performance. In exemplary aspects, of these embodiments, muscle performance is enhanced through increasing potassium (K+) transport into skeletal muscle. In further aspects, muscle performance is enhanced through increasing intracellular calcium (e.g., via ryanodine receptor (RyR) activation).

In certain aspects of the foregoing embodiments wherein the composition comprises effective amounts of dileucine and paraxanthine, the administration of paraxanthine and dileucine produce a synergistic increase in muscle size and/or function in the subject, relative to the administration of paraxanthine or dileucine alone.

In certain further aspects of the foregoing embodiments wherein the composition comprises effective amounts of dileucine and L-arginine, the administration of L-arginine and dileucine produce a synergistic increase in muscle size and/or function in the subject, relative to the administration of L-arginine or dileucine alone.

### Weight Loss

According to certain embodiments, fat loss is promoted through inducing thermogenesis in the subject. According to exemplary implementations of these embodiments, the composition may also include one or more compounds selected from: caffeine, green tea, capsaicin, garcinia cambogia, yohimbine, catechols, epigallocatechin gallate EGCG, catechins, and proanthocyanidins and octacosanol and bitter orange.

According to further embodiments, fat loss is promoted through suppression of appetite in the subject. In exemplary implementations of these embodiments, the composition further may include one or more compounds selected from: fenugreek, glucomannan, gymnema sylvestre, 5-HTP, Caralluma fimbriata, green tea extract, Conjugated linoleic acid, Garcinia cambogia, and Yerba mate.

According to still further embodiments, fat loss is promoted through enhancing lipolysis in the subject. In exemplary implementations of these embodiments, the composition further may include one or more compounds selected from caffeine, green tea extract, L-carnitine, Garcinia cambogia (hydroxycitric acid), capsaicin, ginseng, taurine, silk peptides, BAIBA, grains of paradise, ginger and octacosanol.

In certain embodiments, the disclosed composition, when administered to a subject, increases the subjects resting energy expenditure, relative to the subject's baseline level or following administration of a placebo. In certain embodiments, the increase in the subject's resting energy expenditure following administration of the disclosed compositions is from about 3% to about 30%. In further embodiments, increase in the subject's resting energy expenditure following administration of the disclosed compositions is from about 5% to about 25%. In yet further embodiments increase in the subject's resting energy expenditure following administration of the disclosed compositions is from about 8% to about 20%. In still further embodiments, increase in the subject's resting energy expenditure following administration of the disclosed compositions is from about 10%.

According to certain implementations, the disclosed method further comprises restricting calorie intake of the subject. In exemplary implementations, the amount of fat loss in the subject is greater than that for a subject with an equivalent calorie restriction that has not been provided the composition. According to further implementations, the ratio of fat loss to muscle loss in the subject the subject is greater than that for a subject with an equivalent calorie restriction that has not been provided the composition.

Further disclosed herein are methods for suppressing appetite in a subject by administering to the subject a composition disclosed herein. In certain embodiments, administration of the composition to the subject reduces the subject's appetite by from 5% to about 70%. In further embodiments, reduction of the subject's appetite is from about 10% to about 60%. In yet further embodiments, reduction of the subject's appetite is from about 20% to about 50%. In still further embodiments, reduction of the subject's appetite is at least about 30%.

According to certain embodiments of the disclosed method, the composition is administered in a therapeutically effective amount. In further embodiments, the composition is administered in a prophylactically effective amount.

In certain aspects, disclosed herein are methods to promote weight loss through the administration of an effective amount of one or more compositions disclosed herein. According to certain aspects, administration of effective amounts of the disclosed compositions are used in treating diabetes mellitus; preventing, slowing progression of, delaying or treating of a condition or disorder selected from the group consisting of complications of diabetes mellitus; preventing, slowing the progression of, delaying or treating a metabolic disorder selected from the group consisting of type 1 diabetes mellitus, type 2 diabetes mellitus, impaired glucose tolerance (IGT), impaired fasting blood glucose (IFG), hyperglycemia, postprandial hyperglycemia, overweight, obesity, metabolic syndrome and gestational diabetes; or improving glycemic control and/or for reducing of fasting plasma glucose, of postprandial plasma glucose and/or of glycosylated hemoglobin HbA1c; or preventing, slowing, delaying or reversing progression from impaired glucose tolerance IGT), impaired fasting blood glucose (IFG), insulin resistance and/or from metabolic syndrome to type 2 diabetes mellitus; or preventing, slowing the progression of, delaying or treating of a condition or disorder selected from the group consisting of complications of diabetes mellitus such as cataracts and micro- and macrovascular diseases, such as nephropathy, retinopathy, neuropathy, tissue ischemia, arteriosclerosis, myocardial infarction, stroke and peripheral arterial occlusive disease; or reducing body weight and/or body fat or preventing an increase in body weight and/or body fat or facilitating a reduction in body weight and/or body fat; or preventing, slowing, delaying or treating diseases or conditions attributed to an abnormal accumulation of ectopic fat; or maintaining and/or improving the insulin sensitivity and/or for treating or preventing hyperinsulinemia and/or insulin resistance; preventing, slowing progression of, delaying, or treating new onset diabetes after transplantation (NODAT) and/or post-transplant metabolic syndrome (PTMS); preventing, delaying, or reducing NODAT and/or PTMS associated complications including micro- and macrovascular diseases and events, graft rejection, infection, and death; treating diabetes associated with cystic fibrosis treating hyperuricemia and hyperuricemia associated conditions; treating or prevention kidney stones; treating hyponatremia; in a patient in need thereof.

Another aspect encompasses a combination therapy to regulate fat storage, energy utilization, and/or weight loss in a subject. In an exemplary embodiment, a combination for increasing energy utilization, decreasing body fat or for promoting weight loss may include combining the methods and compositions disclosed with a procedure or therapy such as a pharmaceutical therapy, gastric bypass, duodenojejunal bypass, biliopancreatic diversion, vertical sleeve gastrectomy, adjustable gastric banding, vertical banded gastroplasty, intragastric balloon therapy, gastric plication, Magenstrasse and Mill, small bowel transposition, biliary diversion, brown adipose tissue modulation (e.g., controlled activation, enhanced differentiation, supplemental implantation, etc.), pharmaceutical administration, electrical stimulation of nerves that innervate at least a portion of the gastrointestinal tract, therapies impacting circadian rhythms, bile acid modulation, intestinal mucus production and metabolism, duodenal endoluminal barrier or similar manipulations of the gastrointestinal tract. For example, a composition dileucine can be administered to the subject prior to, concurrently with or after a gastric bypass or other gastrointestinal or bariatric procedure.

In certain aspects, administration of the disclosed compositions is effective at preventing reducing body weight and/or body fat or preventing an increase in body weight and/or body fat or facilitating a reduction in body weight and/or body fat; or preventing, slowing, delaying or treating diseases or conditions attributed to an abnormal accumulation of liver fat; or maintaining and/or improving the insulin sensitivity and/or for treating or preventing hyperinsulinemia and/or insulin resistance.

According to certain embodiments, the administration of dileucine and paraxanthine produces a synergistic improvement in weight management in the subject, relative to the administration of a comparable dose of dileucine and/or paraxanthine alone.

According to further embodiments, wherein the administration of dileucine and L-arginine produces a synergistic improvement in weight management in the subject, relative to the administration of a comparable dose of dileucine and/or L-arginine alone.

### Cognitive Function

Disclosed herein is a method of enhancing cognitive function in a subject comprising administering to the subject a composition disclosed herein. In certain embodiments, improved cognitive function is measured by an increase in one or more of: attention, information acquisition, information processing, working memory, short-term memory, long-term memory, anterograde memory, retrograde memory, memory retrieval, discrimination learning, decision-making, inhibitory response control, attentional set-shifting, delayed reinforcement learning, reversal learning, the temporal integration of voluntary behavior, speed of processing, reasoning, problem solving and/or social cognition.

In certain embodiments, administration of the disclosed composition increases working memory.

In further embodiments, administration of the disclosed composition increases attention.

According to certain embodiments, composition of the instantly disclosed methods to enhance cognitive function further comprise tyrosine, N-acetyl-tyrosine, taurine, huperzine A, acetyl-1-carnitine, CDP choline, Alpha GPC, choline bitrate, choline citrate, B12, caffeine, methyllliberine, theacrine, paraxanthine, theobromine, ashwagandha, rhodiola, lutein, zeaxanthin, fish oil, creatine, ginseng, lions mane, niacin, cordyceps, theanine, B-vitamins, GABA, sulbutiamine, vinpocetine, adenosine triphosphate, inositol, enhanced arginine silicate, nitrates, electrolytes, hesperidin and derivatives of hesperidin and/or bacopa.

According to certain embodiments, the administration of dileucine and paraxanthine produces a synergistic improvement in cognitive function in the subject, relative to the administration of a comparable dose of dileucine and/or paraxanthine alone.

According to further embodiments, wherein the administration of dileucine and L-arginine produces a synergistic improvement in cognitive function in the subject, relative to the administration of a comparable dose of dileucine and/or L-arginine alone.

In certain embodiments, the subject has experience age-related cognitive decline. In exemplary implementations, administration of the composition to the subject increases the level BDNF in the subject. According to certain embodiments, administration of the composition to the subject increases brain derived neurotrophic factor (BDNF) levels in the subject. In exemplary implementations, BDNF levels are increased by from about 5% to about 40%. In further embodiments, BDNF levels are increased by at least about 15%. In further embodiments, administration of the composition to the subject increases other neurotrophic factors such as neuronal growth factor (NGF). In still further embodiments, administration of the composition to the subject increases levels of mTOR in the CNS.

### Enhancing Immune Function

Further disclosed herein, are methods for improving immune function and/or inhibiting inflammation by administering the instantly disclosed compositions to a subject in need thereof. In particular, the disclosed compositions may be used to improve immune function in that the ability to respond to an acute pathogenic stimulus is enhanced. Further, the disclosed composition when administered according to the instantly disclosed methods enhances protective immune response to an exogenous pathogen or to an autologous trigger such as neoplastic cells. Accordingly, the instantly disclosed compositions may be used as a prophylactic, e.g. to avoid an infection or reduce the severity of an infection or another immune-related disease.

In certain embodiments, the disclosed composition is administered to a subject at risk of contracting and infectious disease. In exemplary implementations of these embodiments, the composition may be administered at a prophylactically effective amount.

In further embodiments, the disclosed compositions may be administered to subject who is suffering from an infectious disease or condition. In exemplary implementations of these embodiments, the disclosed composition may be administered in a therapeutically effective amount.

In certain embodiments, the composition also includes one or more additional actives selected from the list consisting of: Cyclosporine, tacrolimus, rapamycin, Omega-3 fatty acids, Curcumin, S-adenosylmethionine, Zinc, Green tea extract, Frankincense (Boswellia serrata resin), Capsaicin, Cat's claw (uncaria plants, including Uncaria tomentosa and Uncaria guianensis), Schizonepeta tenuifolia, Pomegranate, Moringa oleifera, Ecklonia cava, Limonene, Sunifiram, Sulforaphane, Angelica gigas, Ascophyllum nodosum, Scutellaria baicalensis, celery seed extract, Sesamin, Feverfew, Taurine, Rosmarinic Acid, Evodia rutaccarpa, Green Tea Catechins, Punicalagins, Artemisia iwayomogi, Pyrroloquinoline quinone, N-Acetylcysteine, King Oyster, Methylsulfonylmethane, alpha-lipoic acid, pine pollen, Sophora flavescens, Ophiopogon japonicus, Stephania tetrandra, Crataegus pinnatifida, grape seed extract, Bladderwrack, Pacderia foetida, Benfotiamine, Rubus corcanus, Punicic Acid, Sea Buckthorn, Hibiscus rosasinensis, Phellodendron amurense, Resveratrol, Quercetin, Rooibos, Olive leaf extract, Pterostilbene, Eucommia ulmoides, Diindolylmethane, Anatabine, Serrapeptase, Pelargonidin, watercress, Astaxanthin, Piccatannol, Fish Oil, Glutathione, Orthosiphon stamineus, Aronia melanocarpa, blueberry, Tripterygium wilfordii, Boerhaavia diffusa, Whey Protein, Bromelain, Panax ginseng, Aloe vera, cocoa extract, stinging nettle, garlic, Centella asiatica, Astragalus membranaceus, Dendrobium, Vitamin C, Spirulina, Berberine, Ganoderma lucidum, Vitamin C, Vitamin D, Vitamin E, lutein, Pau d'arco, AHCC, rhodiola ashwagandha, shiake, maitake, turkey tail, monolaurin, lysine, Ergothioneine, medium chain triglycerides (MCTs) and butyrate.

According to certain embodiments, the administration of dileucine and paraxanthine produces a synergistic enhancement of immune function in the subject, relative to the administration of a comparable dose of dileucine and/or paraxanthine alone.

According to further embodiments, wherein the administration of dileucine and L-arginine produces a synergistic improvement in enhancement of immune function in the subject, relative to the administration of a comparable dose of dileucine and/or L-arginine alone.

### Anti-Inflammatory

Further disclosed herein is a method of inhibiting inflammation in a subject in need thereof comprising administering to the subject a composition disclosed herein. The method includes administering to a subject in need thereof a therapeutically effective amount of a composition comprising dileucine in an amount of from about 10 mg to about 5,000 mg; and
a) paraxanthine in an amount of from about 2 mg to about 800 mg; or
b) L-arginine in an amount of from about 500 mg to about 6,000 mg.

In certain aspects, paraxanthine is present in the composition in amount from about 20 mg to about 600 mg. In further aspects, paraxanthine is present in the composition in amount from about 50 mg to about 400 mg.

In certain embodiments, the one or more additional active is an anti-inflammatory agent. Disclosed anti-inflammatory agents include agents that exert an anti-inflammatory effect by inhibiting prostaglandins (e.g. targeting COX-2), increasing macrophage mediated phagocytosis (e.g. Resolvins), suppressing cytokine-driven inflammation (e.g., glucocorticoids) suppressing cytokine-driven inflammation, inhibiting cytokines, inhibiting histone deacetylation, inhibiting kinases, stimulating PPAR and/or inhibiting proteases.

Anti-inflammatory agents include, but are not limited to, aspirin, ibuprofen, naproxen, hyssop, ginger, turmeric, helenalin, cannabichromene, rofecoxib, celecoxib, paracetamol (acetaminophen), sirolimus (rapamycin), dexamethasone, dipyridamole, alfuzosin, statins, and glitazones.

In further embodiments, steroidal anti-inflammatory agents, is a nonsteroidal anti-inflammatory agent, or a combination thereof. In some embodiments, anti-inflammatory agents include clobetasol, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone, dexamethasone acetate, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus, prodrugs thereof, co-drugs thereof, and combinations thereof. The anti-inflammatory agent may also be a biological inhibitor of proinflammatory signaling molecules including antibodies to such biological inflammatory signaling molecules.

According to certain embodiments, the administration of dileucine and paraxanthine produces a synergistic inhibition of inflammation in the subject, relative to the administration of a comparable dose of dileucine and/or paraxanthine alone.

According to further embodiments, wherein the administration of dileucine and L-arginine produces a synergistic inhibition of inflammation in the subject, relative to the administration of a comparable dose of dileucine and/or L-arginine alone.

In certain embodiments, the subject is at risk of developing inflammatory disease or condition. In further embodiments, the subject has been diagnosed with an inflammatory disease or condition. In exemplary implementations, the subject has been diagnosed with diabetes, Crohn's disease, rheumatoid arthritis, fibromyalgia, systemic lupus erythematosus, glomerulonephritis, scleroderma, or multiple sclerosis.

In still further embodiments, the one or more additional ingredient is selected from omega-3 fatty acids, vitamin D, vitamin B, protein, selenium, fast digestive carbohydrates like sugar, vitamin K, calcium, vitamin A, ashwagandha (Withania somnifera), Acetylcholine, Acetyl L-Carnitine, tyrosine, N-acetyl-L-tyrosine, Ergothioneine, tryptophan, 5-HTP, arginine, citrulline, norvaline, GABA, Dopa (Velvet Bean), Kanna (serotonin), L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, tetrahydrocurcumin, and Solanum asperum.

### Cardiovascular Health

Further disclosed herein is a method of improving cardiovascular health and/or treating cardiovascular disease or condition by administering a composition disclosed herein. In certain embodiments, improving cardiovascular health as achieved by way of improving blood lipid profile of a subject. As used herein, “improving serum lipid profile” means a reduction is serum total and/or LDL cholesterol and triacyl-glycerol levels, as well as increasing the ratio of HDL cholesterol to LDL cholesterol.

For example, a subject can be identified as having a need for treatment of a disorder (e.g., a disorder related to elevated serum total and/or LDL cholesterol and triacyl-glycerol levels, as well as low ratio of HDL cholesterol to LDL cholesterol) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

In certain aspects, disclosed herein are methods to improving serum lipid profile in a subject through the administration of an effective amount of one or more compositions disclosed herein.

Disclosed herein is a method improving serum lipid profile in a subject in a subject, the method comprises administering to the subject an effective amount of a composition disclosed herein.

In another aspect, a method for preventing or treating a cardiovascular condition, which condition includes a pathology of atherosclerotic plaque formation, is provided. The method includes administering to a subject in need thereof a therapeutically effective amount of a composition disclosed herein. The cardiovascular condition includes, for example, coronary artery disease, coronary microvascular disease, stroke, carotid artery disease, peripheral arterial disease, and chronic kidney disease. The method can include further slowing down the progression of atherosclerotic plaque formation. The method can further include administering one or more additional therapeutic agent to the subject for the prevention or treatment of the cardiovascular condition.

In another aspect, a method for treating metabolic syndrome is provided. The method includes administering to a subject in need thereof a therapeutically effective amount of a composition disclosed herein. The method can further include reducing one or more risk factors associated with metabolic syndrome, including one or more of abdominal obesity, hyperglycemia, dyslipidemia, and hypertension. The method can further include administering one or more additional agent to the subject for the prevention or treatment of metabolic syndrome.

In another aspect, a method for delaying or slowing down the progression of atherosclerosis is provided. The method includes administering to a subject in need thereof a therapeutically effective amount of a composition comprising dileucine in an amount of from about 10 mg to about 5,000 mg; and
a) paraxanthine in an amount of from about 2 mg to about 800 mg; or
b) L-arginine in an amount of from about 500 mg to about 6,000 mg.

In certain aspects, the composition further comprises one or more additional active ingredient to further improving serum lipid profile in a subject. In certain embodiments, the one or more additional active ingredient is an amino acid. According to certain embodiments, the amino acid is selected from a group of branched-chain amino acids (BCAA), including, but not limited to, isoleucine, leucine, and valine. In further embodiments, the amino acid is selected from the group of essential amino acids, including, but not limited to, histidine, isoleucine, leucine, lysine, methionine, phenylalanine, threonine, tryptophan, and valine. In still further embodiments, the amino acid is selected from the group of conditionally essential amino acids including, but not limited to, arginine, cysteine, glutamine, glycine, proline, and tyrosine. According to the certain embodiments, the conditionally essential amino acid is tyrosine. In still further embodiments, the amino acid is selected from the group of non-essential amino acids including, but not limited to, alanine, aspartic acid, asparagine, glutamic acid, serine, selenocysteine and pyrrolysine. In yet further embodiments, the amino acid derivative is selected from the group of creatine, carnitine, creatinol, beta-alanine, taurine, beta-hydroxy beta-methylbutyrate L-Arginine, omega-3 fatty acids, Vitamin D, Non-Steroidal Anti-Inflammatory Drugs (NSAID), whey protein, and other protein extracts from animal, plant or fermentation sources.

In still further embodiments, the one or more additional ingredient is selected from omega-3 fatty acids, vitamin D, vitamin B, protein, selenium, fast digestive carbohydrates like sugar, vitamin K, calcium, vitamin A, ashwagandha (Withania somnifera), Acetylcholine, Acetyl L-Carnitine, tyrosine, N-acetyl-L-tyrosine, Ergothioneine, tryptophan, 5-HTP, arginine, citrulline, norvaline, GABA, Dopa (Velvet Bean), Kanna (serotonin), L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, tetrahydrocurcumin, and Solanum asperum.

According to certain embodiments, the administration of dileucine and paraxanthine produces a synergistic improvement in cardiovascular health in the subject, relative to the administration of a comparable dose of dileucine and/or paraxanthine alone.

According to further embodiments, wherein the administration of dileucine and L-arginine produces a synergistic improvement in cardiovascular health in the subject, relative to the administration of a comparable dose of dileucine and/or L-arginine alone.

### Improving Blood Flow

Further disclosed herein are method of improving blood flow in a subject by administering to the subject a composition disclosed herein. In certain implementations, the disclosed methods for improving blood flow are useful in conjunction with athletic training or performance, in that increased blood flow to the muscle during physical activity can yield increased performance. In further embodiments, the disclosed methods to increase blood flow are useful in the treatment of a disease or condition characterized by reduced blood flow to a tissue or organ system. For example, methods are disclosed herein for promoting blood flow in an area in a subject who has or is at risk for developing ischemia, for instance during or following surgery, burn injury, a graft, peripheral vascular disease, amputation, coronary artery disease, stroke, thrombosis, a clot, chronic vascular obstruction or vasculopathy (e.g., secondary to diabetes, hypertension, or peripheral vascular disease), cerebral ischemia, a wound, and so forth. Provided methods are useful in the treatment of various diseases and conditions, including but not limited to treatment of donor organs before and after transplantation; reattachment of severed extremities, body parts or soft tissues: pulmonary hypertension (adult or neonate); sickle cell disease; neointimal hyperplasia or restenosis (following angioplasty or stenting); primary burn care (before skin grafting): kidney disease (for instance, to increase kidney circulation); preeclampsia; erectile dysfunction; asthma or adult respiratory distress syndrome.

According to certain embodiments, the administration of dileucine and paraxanthine produces a synergistic improvement in blood flow in the subject, relative to the administration of a comparable dose of dileucine and/or paraxanthine alone.

According to further embodiments, wherein the administration of dileucine and L-arginine produces a synergistic improvement in blood flow in the subject, relative to the administration of a comparable dose of dileucine and/or L-arginine alone.

### Routes of Administration

The administration of the disclosed compositions to a subject may include any method of providing a pharmaceutical and/or nutraceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral, administration, rectal administration, sublingual administration, intradermal administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. According to further embodiments, the disclosed compositions may be administered as a beverage, functional food, and/or medicinal food, as described above.

Various aspects and embodiments of the present invention are defined by the following numbered clauses:

1. A composition comprising: paraxanthine and dileucine.

2. The composition of clause 1, wherein paraxanthine is present in an amount of from about 2 mg to about 800 mg.

3. The composition of clause 2, wherein paraxanthine is present in an amount from about 20 mg to about 600 mg.

4. The composition of clause 3, wherein paraxanthine is present in an amount from about 50 mg to about 400 mg.

5. The composition of any of clauses 1-4, wherein dileucine is present in an amount of from about 200 mg to about 5,000 mg.

6. The composition of clause 5, wherein dileucine is present in an amount of from about 500 mg to about 3,000 mg.

7. A composition comprising: dileucine and L-arginine.

8. The composition of clause 7, wherein dileucine is present in an amount of from about 10 mg to about 5,000 mg.

9. The composition of clause 8, wherein dileucine is present in an amount of from about 25 mg to about 2,500 mg.

10. The composition of any of clauses 7-9, wherein L-arginine is present in an amount of about 500 mg to about 6,000 mg.

11. The composition of clause 10, wherein L-arginine is present in an amount of about 1,000 mg to about 3,000 mg.

12. The composition of any of clauses 7-11, wherein the L-arginine is in the form of a free form, salt, bonded arginine silicate complex, and/or arginine alpha-ketoglutarate.

13. The composition of any of clauses 1-12, wherein the composition is a dietary supplement.

14. The composition of any of clauses 1-13, wherein the composition is a powder.

15. The composition of any of clauses 1-13, wherein the composition is a functional food.

16. The composition of clause 15, wherein the functional food is a beverage, nutrition bar, yoghurt, or cereal.

17. The composition of clause 16, wherein the composition is a beverage.

18. The composition of any clauses of 1-12, wherein the composition is a sublingual spray.

19. The composition of any of clauses 1-12, further comprising a nutraceutically acceptable carrier.

20. The composition of clause 19, wherein the nutraceutically acceptable carrier is a capsule.

21. A method of improving muscle strength, muscle size, and/or muscle function comprising: administering to a subject the composition of any of clauses 1-20.

22. The method of clause 21, wherein the composition further comprises one or more compounds selected from the list consisting of: isoleucine, leucine, and valine, histidine, lysine, methionine, phenylalanine, threonine, tryptophan, valine, creatine, cysteine, glutamine, glycine, proline, tyrosine, carnitine, beta-alanine, taurine, beta-hydroxy beta-methylbutyrate, Omega-3 fatty acids, Vitamin D, whey protein, and other protein extracts from animal, plant or fermentation sources.

23. The method of either of clauses 21-22, wherein the composition administered is the composition of any of clauses 1-9, and wherein the administration of dileucine and paraxanthine produces a synergistic improvement in muscle strength, muscle size, and/or muscle function in the subject, relative to the administration of a comparable dose of dileucine and/or paraxanthine alone. 24. The method of either of clauses 21-22, wherein the composition administered is the composition of clauses 10-19, and wherein the administration of dileucine and L-arginine produces a synergistic improvement in muscle strength, muscle size, and/or muscle function in the subject, relative to the administration of a comparable dose of dileucine and/or L-arginine alone.

25. A method for increasing athletic performance in a subject in need thereof, comprising: administering to a subject an effective amount of the composition of any of clauses 1-20.

26. The method of clause 25, wherein the composition administered is the composition of clauses 1-9, and wherein the administration of dileucine and paraxanthine produces a synergistic increase in athletic endurance in the subject, relative to the administration of a comparable dose of dileucine and/or paraxanthine alone.

27. The method of clause 25, wherein the composition administered is the composition of clauses 10-19, and wherein the administration of dileucine and L-arginine produces a synergistic increase in athletic performance in the subject, relative to the administration of a comparable dose of dileucine and/or L-arginine alone.

28. A method for promoting weight loss in a subject, comprising: administering to the subject the composition of any of clauses 1-20.

29. The method of clause 28, wherein weight loss is promoted through inducing thermogenesis in the subject.

30. The method of clause 29, wherein the composition further comprises one or more compounds selected from a list consisting of: caffeine, green tea, capsaicin, garcinia cambogia, yohimbine and bitter orange.

31. The method of clause 28, wherein weight loss is promoted through suppression of appetite in the subject and wherein administration of the composition to the subject suppresses appetite in the subject by at least about 30%.

32. The method of clause 28, wherein weight loss is promoted through enhancing lipolysis in the subject.

33. The method of clause 28, wherein administration of the composition to the subject decreases the respiratory quotient in the subject by at least about 10%.

34. The method of clause 28, wherein resting energy expenditure in the subject is increased by at least about 15%.

35. The method of clause 32, wherein the wherein the composition further comprises one or more compounds selected from a list consisting of caffeine, green tea extract, L-carnitine, Garcinia cambogia (hydroxycitric acid), capsaicin, ginseng, taurine, silk peptides, catechols, epigallocatechin gallate (EGCG), catechins, proanthocyanidins and octacosanol.

36. The method of clause 28, further comprising restricting calorie intake of the subject and wherein the amount of weight loss in the subject is greater than that for a subject with an equivalent calorie restriction that has not been provided the composition and wherein the ratio of fat loss to muscle loss in the subject is greater than that for a subject with an equivalent calorie restriction that has not been provided the composition.

37. The method of clause 28, wherein the subject is not administered caffeine.

38. A method for suppressing appetite in a subject comprising: administering to the subject the composition of any of clauses 1-20.

39. The method of clause 38, wherein administration of the composition decreases appetite in the subject by at least about 30%.

40. The method of clause 38, wherein the subject is not administered caffeine.

41. A method for promoting fat loss in a subject, comprising: administering to the subject with the composition of any of clauses 1-20.

42. The method of clause 41, wherein the subject is not administered caffeine.

43. The method of clause 41, wherein fat loss is promoted through inducing thermogenesis in the subject.

44. The method of clause 43, wherein the composition further comprises one or more compounds selected from the list consisting of: caffeine, green tea, capsaicin, garcinia cambogia, yohimbine, catechols, EGCG, catechins, and proanthocyanidins and octacosanol and bitter orange. 45. The method of clause 41, wherein fat loss is promoted through suppression of appetite in the subject.

46. The method of clause 45, wherein administration of the composition to the subject suppresses appetite in the subject by at least about 30%.

47. The method of clause 41, wherein administration of the composition to the subject decreases the respiratory quotient in the subject by at least about 10%.

48. The method of clause 45, wherein the composition further comprises one or more compounds selected from the list consisting of: fenugreek, glucomannan, gymnema sylvestre, 5-HTP, Caralluma fimbriata, green tea extract, conjugated linoleic acid, Garcinia cambogia, and Yerbamate.

49. The method of clause 41, wherein fat loss is promoted through enhancing lipolysis in the subject.

50. The method of clause 49, wherein the wherein the composition further comprises one or more compounds selected from the list consisting of caffeine, green tea extract, L-carnitine, Garcinia cambogia (hydroxycitric acid), capsaicin, ginseng, taurine, silk peptides and octacosanol.

51. The method of clause 41, further comprising restricting calorie intake of the subject. 52. The method of clause 51, wherein the amount of fat loss in the subject is greater than that for a subject with an equivalent calorie restriction that has not been provided the composition.

53. The method of clause 51, wherein the ratio of fat loss to muscle loss in the is greater than that for a subject with an equivalent calorie restriction that has not been provided the composition. 54. A method of enhancing attention in a subject in need thereof comprising administering the composition of any of clauses 1-20.

55. A method of improving working memory in a subject in need thereof comprising administering a composition to the subject comprising the composition of any of clauses 1-20.

56. A method of improving cognitive performance in a subject comprising administering the composition of any of clauses 1-20.

57. The method of clause 56, wherein improved cognitive function is measured by an increase in one or more of: attention, information acquisition, information processing, working memory, short-term memory, long-term memory, anterograde memory, retrograde memory, memory retrieval, discrimination learning, decision-making, inhibitory response control, attentional set-shifting, delayed reinforcement learning, reversal learning, the temporal integration of voluntary behavior, speed of processing, reasoning, problem solving and/or social cognition.

58. The method of clauses 56-57, wherein the composition administered is the composition of clauses 1-9, and wherein the administration of dileucine and paraxanthine produces a synergistic improvement in cognitive function in the subject, relative to the administration of a comparable dose of dileucine and/or paraxanthine alone.

59. The method of clauses 56-57, wherein the composition administered is the composition of clauses 10-19, and wherein the administration of dileucine and L-arginine produces a synergistic improvement in cognitive function in the subject, relative to the administration of a comparable dose of dileucine and/or L-arginine alone.

60. A method of treating an inflammatory disease or condition in a subject in need thereof comprising administering to the subject a composition comprising the composition of any of clauses 1-20.

61. The method of clause 60, wherein the composition further comprises a further anti-inflammatory agent.

62. The method of clause 61, wherein the second anti-inflammatory agent exerts anti-inflammatory effects by: inhibiting prostaglandins, increasing macrophage mediated phagocytosis, suppressing cytokine-driven inflammation, inhibiting cytokines, inhibiting histone deacetylation, inhibiting kinases, stimulating PPAR and/or inhibiting proteases.

63. The method of clause 61, wherein the further anti-inflammatory agent is selected from aspirin, ibuprofen, naproxen, hyssop, ginger, turmeric, helenalin, cannabichromene, rofecoxib, celecoxib, paracetamol (acetaminophen), sirolimus (rapamycin), dexamethasone, dipyridamole, alfuzosin, statins, and glitazones.

64. A method for improving joint health in a subject in need thereof comprising:

providing the subject with a composition comprising the composition of any of clauses 1-20.

65. The method of clause 64, wherein the subject has been diagnosed with an inflammatory joint disease or condition.

66. The method of clause 64, wherein the subject is at risk of developing inflammatory joint disease or condition.

67. A method of treating an inflammatory joint disease comprising: administering to the subject a composition comprising the composition of any of clauses 1-20.

68. The method of clause 67, wherein the composition is administered in a therapeutically effective amount.

69. The method of clause 67, wherein the composition is administered in a prophylactically effective amount.

70. A method for enhancing immune function in a subject, comprising:
providing the subject with a composition comprising the composition of any of clauses 1-20.

71. The method of clauses 70, wherein the composition administered is the composition of clauses 1-9, and wherein the administration of dileucine and paraxanthine produces a synergistic enhancement of immune function in the subject, relative to the administration of a comparable dose of dileucine and/or paraxanthine alone.

72. The method of clauses 70, wherein the composition administered is the composition of clauses 10-19, and wherein the administration of dileucine and L-arginine produces a synergistic enhancement of immune function in the subject, relative to the administration of a comparable dose of dileucine and/or L-arginine alone.

73. The method of any of clauses 70-72, wherein the composition further comprises one or more compounds selected from the list consisting of: Cyclosporine, tacrolimus, rapamycin, Omega-3 fatty acids, Curcumin, S-adenosylmethionine, Zinc, Green tea extract, Frankincense (Boswellia serrata resin), Capsaicin, Cat's claw (uncaria plants, including Uncaria tomentosa and Uncaria guianensis), Schizonepeta tenuifolia, Pomegranate, Moringa oleifera, Ecklonia cava, Limonene, Sunifiram, Sulforaphane, Angelica gigas, Ascophyllum nodosum, Scutellaria baicalensis, celery seed extract, Sesamin, Feverfew, Taurine, Rosmarinic Acid, Evodia rutaecarpa, Green Tea Catechins, Punicalagins, Artemisia iwayomogi, Pyrroloquinoline quinone, N-Acetylcysteine, King Oyster, Methylsulfonylmethane, alpha-lipoic acid, pine pollen, Sophora flavescens, Ophiopogon japonicus, Stephania tetrandra, Crataegus pinnatifida, grape seed extract, Bladderwrack, Pacderia foctida, Benfotiamine, Rubus corcanus, Punicic Acid, Sea Buckthorn, Hibiscus rosasinensis, Phellodendron amurense, Resveratrol, Quercetin, Rooibos, Olive leaf extract, Pterostilbene, Eucommia ulmoides, Diindolylmethane, Anatabine, Serrapeptase, Pelargonidin, watercress, Astaxanthin, Piccatannol, Fish Oil, Glutathione, Orthosiphon stamineus, Aronia melanocarpa, blueberry, Tripterygium wilfordii, Boerhaavia diffusa, Whey Protein, Bromelain, Panax ginseng, Aloe vera, cocoa extract, stinging nettle, garlic, Centella asiatica, Astragalus membranaceus, Dendrobium, Vitamin C, Spirulina, Berberine, Ganoderma lucidum, Vitamin C, Vitamin D, Vitamin E, lutein, leucine, dileucine, trileucine, tetraleucine Pau d'arco, AHCC, rhodiola ashwagandha, shiake, maitake, turkey tail, monolaurin, lysine, Ergothioncine, medium chain triglycerides (MCTs) and butyrate.

74. A method for improving cardiovascular health in a subject, comprising: providing the subject with a composition comprising the composition of any of clauses 1-20.

75. The method of clauses 74, wherein the composition administered is the composition of clauses 1-9, and wherein the administration of dileucine and paraxanthine produces a synergistic improvement in cardiovascular health in the subject, relative to the administration of a comparable dose of dileucine and/or paraxanthine alone.

76. The method of clauses 74, wherein the composition administered is the composition of clauses 10-19, and wherein the administration of dileucine and L-arginine produces a synergistic improvement in cardiovascular health in the subject, relative to the administration of a comparable dose of dileucine and/or L-arginine alone.

77. A method for improving blood flow in a subject, comprising: providing the subject with a composition comprising the composition of any of clauses 1-20.

78. The method of clauses 77, wherein the composition administered is the composition of clauses 1-9, and wherein the administration of dileucine and paraxanthine produces a synergistic improvement in blood flow in the subject, relative to the administration of a comparable dose of dileucine and/or paraxanthine alone.

79. The method of clauses 77, wherein the composition administered is the composition of clauses 10-19, and wherein the administration of dileucine and L-arginine produces a synergistic improvement in blood flow in the subject, relative to the administration of a comparable dose of dileucine and/or L-arginine alone.

80. A method for increasing athletic endurance in a subject, comprising: administering to a subject an effective amount of the composition of any of clauses 1-20.

81. The method of clause 80, wherein the composition administered is the composition of one of clauses 1-9, and wherein the administration of dileucine and paraxanthine produces a synergistic increase in athletic endurance in the subject, relative to the administration of a comparable dose of dileucine and/or paraxanthine alone.

82. The method of clause 80, wherein the composition administered is the composition one of clauses 10-19, and wherein the administration of dileucine and L-arginine produces a synergistic increase in athletic endurance in the subject, relative to the administration of a comparable dose of dileucine and/or L-arginine alone.

### EXPERIMENTAL

### Examples

The following examples are put forth so as to provide those of ordinary skill in the art with a complete disclosure and description of certain examples of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

### Animals and Experiment Design

Eight 8-week-old male Swiss Albino mice per experimental group were housed in an animal room at a constant temperature (22±3° C.) and humidity (30%-70%) under a 12:12 h light-dark cycle with standard laboratory diet (Purina 5L79, Rat and Mouse 18% protein; PMI Nutrition International, Brentwood, MO, USA). Distilled water was provided ad libitum. All animal experiments were reviewed and approved by the Institutional Animal Ethical Committee (IAEC) of Radiant Research Services Pvt. Ltd (Bangalore, India). All research was conducted in accordance with the guidelines of the committee for the purpose of control and supervision of experiments on animals (CPCSEA Registration Number-1803/PO/RcBi/S/2015/CPCSEA).

After one week of acclimation, the animals were randomly divided by body weight into different groups (n=8 per group in each test) for oral treatment once a day, at approximately same time each day (+1 hour), for 28 consecutive days: (1) vehicle control; (2) paraxanthine (3) dileucine; (4) paraxanthine+dileucine; (5) L-arginine; (6) L-arginine+dileucine; (7) L-citrulline; and (8) L-citrulline+dileucine. The dose administered to the mice was calculated using US Food and Drug Administration for human equivalence doses (HED), assuming a human weight of 60 kg. The following HED were used in this study: 2,000 mg dileucine (RAMPS™, Ingenious Ingredients, L.P., Lewisville, TX, USA; mouse 411 mg/kg bw), 100 mg paraxanthine, (ENFINITY™, Ingenious Ingredients, L.P Lewisville, TX, USA; mouse dose: 20.5 mg/kg bw/day); 3,000 mg L-arginine (Nanjing Nutrabuilding Bio-tech Co., LTD., No. 270 Jiqingmen Strect, Suning Huigu, Building E6, Room 2105, Nanjing, China 210017; mouse 616.55 mg/kg bw), 3,000 mg L-citrulline (Nanjing Nutrabuilding Bio-tech Co., LTD., No. 270 Jiqingmen Street, Suning Huigu, Building E6, Room 2105, Nanjing, China 210017; mouse 616.55 mg/kg bw). 0.5% Carboxy Methyl Cellulose sodium was used as vehicle and the test item formulations were prepared daily. Dosing was conducted via oral gavage using disposable polypropylene syringes with sterilized stainless steel gavage tubes. The food intake and water consumption were monitored daily, and BW was recorded weekly. The last dose on day 28 was given 1 hour prior to testing.

### Sample Collection

All animals were euthanized by 95% CO2 after 28 days of following their assigned treatment. Blood was collected immediately after euthanization by the retro-orbital route and was immediately centrifuged at 1500×g for 10 min at 4° C. before having all serum transferred into cryogenic tubes and stored at −80° C.

### Forelimb Grip Strength Test

The forelimb grip strength was measured on day 0 and day 28 by using a stainless-steel grill to assess muscle strength (Orchid Scientific & Innovative India Pvt Ltd, India). Grip strength was measured one hour after treatment. Briefly, each mouse was placed on a grill chamber and the steel bar is inverted; grip strength is measured by recording the time each animal holds the steel bar. The maximum time for each trial was six minutes. Each animal performed three independent trials separated by six minutes intervals and the mean of the three trials was calculated.

### Exercise Training

During the treatment period, exercise training was completed using a motorized treadmill (Exer 3/6, Columbus Instruments international, OH, USA) at a moderate intensity of 20 cm/sec as maximal running speed, an incline of ten degrees and a shock intensity of 0.2 mA, for ten minutes. The speed of the treadmill was manually adjusted by increasing the belt speed by 5 cm/sec every two minutes throughout the total duration of ten minutes. All animals were adapted to this procedure daily 60 minutes after dosing for five days in a week during the treatment period.

### Treadmill Endurance Test

On 28th day of each respective treatment, all animals were subjected to a muscle endurance test. Muscle endurance was accomplished on a motorized treadmill at a low to moderate intensity of 5-50 cm/sec as maximal running speed, an incline of ten degrees and a shock intensity of 0.2 mA, with the belt speed being increased by 5 cm/sec every two minutes until it reaches 50 cm/sec. Animals were subjected to the treadmill test until exhaustion. Distance traveled (m) was measured as a marker of exercise performance.

### Body Weight

Individual body weights were recorded at receipt, on the day of randomization, on the first day of treatment before dosing (Day 1) and weekly thereafter. The body weight changes for all the animals were calculated and reported along with the body weight data.

### Biochemical Parameters

Serum and plasma samples were separated by centrifuging the blood at 8000 rpm for 10 mins and analyze the below mentioned biomarkers, Biomarkers: TC, TG, LDL and HDL, CRP, Serum lactate, Ammonia, Glucose, NO, IL-2, IL-6, IL-10. Blood was collected in EDTA tubes for below mentioned biomarkers, Immune health Biomarkers (T helper subsets): TH1, TH2, TH17, Tfh, iTreg.

### Statistical Analysis

Data are presented as mean±standard deviation (SD). Statistical differences among groups were analyzed using one-way ANOVA followed by the Dunnett's Test identify the pairs with significant differences using GraphPad PRISM Software, Version5.01 (GraphPad Software, Inc. California, USA). The level of statistical significance was set at p<0.05.

### Results

### Effect on Body Weight and Feed Consumption

The initial and final body weights and food consumption did significantly differ between groups. Combinations of dileucine with paraxanthine, L-arginine, and L-citrulline showed synergetic effects over control. The daily feed consumption was kept constant between groups over 28 days. The combination of paraxanthine and dileucine showed synergistic effects over the effect of the single substances, resulting in leaner mice after 28 days. The body weight changes are summarized in Table 1.

TABLE 1
Effect of test substance on body weight (in grams) and changes
(in percentages). Data for n = 8 mice in each group.

| | | | | Percentage |
|---|---|---|---|---|
| | Body Weight (grams) | | Weight gain | weight loss |
| Treatment | Day −0 | Day −28 | in grams | over control |
| Exercise Control | 23.4 | 34.15 | 10.75 | |
| Dileucine | 24.09 | 33.23* | 9.14 | −15 |
| Paraxanthine | 24.35 | 33.21* | 8.86 | −18 |
| Dileucine + | 24.31 | 32.29*** | 7.98 | −26 |
| Paraxanthine | | | | |
| L-Arginine | 23 | 33.01* | 10.01 | −7 |
| Dileucine + | 22.8 | 32.63*** | 10.04 | −7 |
| L-Arginine | | | | |
| L-Citrulline | 22.49 | 33.01* | 9.83 | −9 |
| Dileucine + | 23.1 | 32.76** | 9.66 | −10 |
| L-Citrulline | | | | |

Data represented by Mean ± SD (n = 8 in each group). Statistical significances are compared between Exercise Control (G1) versus other treatment groups
(*P Value < 0.05);
(**P Value < 0.01);
(***P Value < 0.001)

### Effect of Supplementation on Forelimb Grip Strength

The initial and final forelimb grip strength did significantly differ for all groups compared to exercise control. Combinations of dileucine with paraxanthine, L-arginine, and L-citrulline showed synergetic effects over control. The combination of paraxanthine and dileucine excelled as synergistic pairing. The forelimb grip strength changes are summarized in Table 2.

TABLE 2
Effect of test substance on forelimb grip strength (in gf) and
changes (in percentages).Data for n = 8 mice in each group.

| | | | Increase | Percentage |
|---|---|---|---|---|
| | Forelimb grip strength (gf) | | strength | Increase |
| Treatment | Day −0 | Day −28 | over Day −0 | over control |
| Exercise | 98.88 | 127.13 | 28.25 | |
| Control | | | | |
| Dileucine | 99.25 | 149.0*** | 49.75 | 76 |
| Paraxanthine | 98.38 | 148.88*** | 50.5 | 79 |
| Dileucine + | 100.63 | 153.63*** | 53 | 88 |
| Paraxanthine | | | | |
| L-Arginine | 99.75 | 139.13*** | 39.38 | 39 |
| Dileucine + | 97.75 | 149.63*** | 51.88 | 84 |
| L-Arginine | | | | |
| L-Citrulline | 97.63 | 138.5*** | 40.87 | 45 |
| Dileucine + | 99.35 | 142.0*** | 49.65 | 76 |
| L-Citrulline | | | | |

Data represented by Mean ± SD (n = 8 in each group). Statistical significances are compared between Exercise Control (G1) versus other treatment groups
(*P Value < 0.05);
(**P Value < 0.01);
(***P Value < 0.001)

Effect of Supplementation on Number of Shocks during Treadmill Exercise

The initial and final number of shocks during treadmill exercise did differ for all groups compared to exercise control. Combinations of dileucine with paraxanthine, L-arginine, and L-citrulline showed synergetic effects over control. The combination of paraxanthine and dileucine excelled as synergistic pairing. The forelimb grip strength changes are summarized in Table 3.

TABLE 3
Effect of test substance on number shocks during
treadmill performance (in numbers) and changes (in
percentages). Data for n = 8 mice in each group.

| | No. of shocks during | | No of Shocks | Percentage |
|---|---|---|---|---|
| | treadmill exercise | | Decrease over | Increase |
| Treatment | Day −0 | Day −28 | Day −0 | over control |
| Exercise Control | 28.75 | 20.88 | −7.87 | |
| Dileucine | 27.63 | 13*** | −14.63 | 86 |
| Paraxanthine | 26.5 | 13.38*** | −13.12 | 67 |
| Dileucine + | 29.88 | 9.38*** | −20.5 | 160 |
| Paraxanthine | | | | |
| L-Arginine | 30.38 | 16.75*** | −13.63 | 73 |
| Dileucine + | 27.38 | 13.13*** | −14.25 | 81 |
| L-Arginine | | | | |
| L-Citrulline | 28.63 | 17.00*** | −11.63 | 48 |
| Dileucine + | 27.5 | 14.13*** | −13.37 | 70 |
| L-Citrulline | | | | |

Data represented by Mean ± SD (n = 8 in each group). Statistical significances are compared between Exercise Control (G1) versus other treatment groups
(*P Value < 0.05);
(**P Value < 0.01);
(***P Value < 0.001)

Effect of Supplementation on Time Spent on Shock Zone during Treadmill Exercise The initial and final number of times spent of the shock zone during treadmill exercise did differ for all groups compared to exercise control. Combinations of dileucine with paraxanthine, L-arginine, and L-citrulline showed synergetic effects over control. The combination of paraxanthine and dileucine excelled as synergistic pairing. The times spent on shock zone during treadmill exercise are summarized in Table 4.

TABLE 4
Effect of test substance on time spent on shock zone
during treadmill performance (in seconds) and changes
(in percentages). Data for n = 8 mice in each group.

| | | | Time spent | Percentage |
|---|---|---|---|---|
| | Time spent on shock zone | | on shock | Increase |
| | during excercise [sec] | | zone over | over |
| Treatment | Day −0 | Day −28 | Day −0 | control |
| Exercise | 90.13 | 59.63 | −30.5 | |
| Control | | | | |
| Dileucine | 91 | 31.88*** | −59.12 | 94 |
| Paraxanthine | 90.75 | 32.25*** | −58.5 | 92 |
| Dileucine + | 93.75 | 27.63*** | −66.12 | 117 |
| Paraxanthine | | | | |
| L-Arginine | 90 | 35.13*** | −54.87 | 80 |
| Dileucine + | 90.5 | 32.0*** | −58.5 | 92 |
| L-Arginine | | | | |
| L-Citrulline | 90.88 | 36.63*** | −54.25 | 78 |
| Dileucine + | 89.75 | 32.88*** | −56.87 | 86 |
| L-Citrulline | | | | |

Data represented by Mean ± SD (n = 8 in each group). Statistical significances are compared between Exercise Control (G1) versus other treatment groups
(*P Value < 0.05);
(**P Value < 0.01);
(***P Value < 0.001)

Effect of Supplementation on Distance Travelled during Treadmill Exercise The initial and final number of distances travelled during treadmill exercise did significantly differ for all groups compared to exercise control. Combinations of dileucine with paraxanthine, L-arginine, and L-citrulline showed synergetic effects over control. The combination of paraxanthine and dileucine excelled as synergistic pairing. The distances travelled during treadmill exercise are summarized in Table 5.

TABLE 5
Effect of test substance on distance travelled during exercise (in meter)
and changes (in percentages). Data for n = 8 mice in each group.

| | | |
|---|---|---|
| Distance travelled | Distance | Percentage |
| (m) by mice during | Increase | Increase |
| treadmill exercise | over | over |

| Treatment | Day −0 | Day −28 | Day −0 | control |
|---|---|---|---|---|
| Exercise | 40.25 | 179.13 | 138.88 | |
| Control | | | | |
| Dileucine | 38.88 | 328.5*** | 289.62 | 109 |
| Paraxanthine | 41.13 | 325.75*** | 284.62 | 105 |
| Dileucine + | 38.25 | 346.88*** | 308.63 | 122 |
| Paraxanthine | | | | |
| L-Arginine | 39.13 | 314.25*** | 275.12 | 98 |
| Dileucine + | 38.38 | 336.88*** | 298.5 | 115 |
| L-Arginine | | | | |
| L-Citrulline | 38.5 | 312*** | 273.5 | 97 |
| Dileucine + | 39.38 | 330.5*** | 291.12 | 110 |
| CL-itrulline | | | | |

Data represented by Mean ± SD (n = 8 in each group). Statistical significances are compared between Exercise Control (G1) versus other treatment groups
(*P Value < 0.05);
(**P Value < 0.01);
(***P Value < 0.001)

### Effect on Blood Lipids During Treadmill Exercise

The initial numbers of the blood lipid measurements show the impact on the synergistic combination of paraxanthine and dileucine on total cholesterol (TC), triglycerides (TG), high-density lipids (HDL), and low-density lipids (LDL) over control after 28 days of supplementation. The blood lipid measurements are shown in table 6.

TABLE 6
Effect of test substance on blood lipids. Data for n = 8 mice in each group.

| | | Change | | Change | | Change | | Change |
|---|---|---|---|---|---|---|---|---|
| | | over | | over | | over | | over |
| | | control | | control | HDL | control | LDL | control |
| Treatment | TC [mg/dl] | [%] | TG [mg/dl] | [%] | [mg/dl] | [%] | [mg/dl] | [%] |
| Exercise Control | 99.4 | | 95.36 | | 34.9 | | 43.49 | |
| Dileucine | 93.3 | −6 | 92.66 | −3 | 37.63* | 8 | 42.05 | −3 |
| Paraxanthine | 91.66** | −8 | 91.94 | −4 | 36.36 | 4 | 43.23 | −1 |
| Dileucine + | 88.3*** | −11 | 89.1 | −7 | 37.93** | 9 | 39.84 | −8 |
| Paraxanthine | | | | | | | | |
| L-Arginine | 95.43 | −5 | 94.1 | −2 | 35.86 | 5 | 44.66 | 3 |
| Dileucine + L- | 93.08** | −7 | 93.98 | −2 | 36.54* | 7 | 41.95 | −4 |
| Arginine | | | | | | | | |
| L-Citrulline | 94.26* | −6 | 94.58 | −2 | 35.63 | 4 | 44.01 | 1 |
| Dileucine + L- | 92.81** | −7 | 94.26 | −2 | 36.1 | 5 | 43.08 | −1 |
| Citrulline | | | | | | | | |

Data represented by Mean ± SD (n = 8 in each group). Statistical significances are compared between Exercise Control (G1) versus other treatment groups
(*P Value < 0.05);
(**P Value < 0.01);
(***P Value < 0.001)

### Effect of Supplementation on Inflammatory Markers and NO Signaling

The synergistic combination of paraxanthine+dileucine, and L-arginine+dileucine significantly increased IL-2, IL-10, and nitric oxide, and significantly decreased IL-6 and C-reactive protein (CRP) over control after 28 days of supplementation and exercise. The inflammatory markers and nitric oxide measurements are shown in table 7.

TABLE 7
Effect on nitric oxide levels. Data are the mean ± SD for n = 8 mice in each group.

| | | Change | | Change | | Change | | Change | Nitric | Change |
|---|---|---|---|---|---|---|---|---|---|---|
| | | over | | over | | over | | over | oxide | over |
| | | control | IL-6 | control | IL-10 | control | CRP | control | (μmol/L) | control |
| Treatment | IL-2 (ng/L) | [%] | (pg/ml) | [%] | (pg/ml) | [%] | (mg/L) | [%] | (mg/L) | [%] |
| Exercise Control | 37.29 | | 369.04 | | 527.86 | | 0.77 | | 63.49 | |
| Dileucine | 46.4*** | 24 | 343** | −7 | 569.36*** | 8 | 0.63*** | −18 | 84.83*** | 34 |
| Paraxanthine | 45.91 | 23 | 340.6** | −8 | 571.71*** | 8 | 0.64** | −17 | 84.29*** | 33 |
| Dileucine + | 50.07*** | 34 | 335.6*** | −9 | 580.26** | 10 | 0.59*** | −23 | 87.8*** | 38 |
| Paraxanthine | | | | | | | | | | |
| L-Arginine | 42.04*** | 23 | 351.39** | −7 | 565.47** | 5 | 0.69*** | −16 | 77.98* | 29 |
| Dileucine + L- | 45.63*** | 33 | 344.4** | −9 | 572.96*** | 7 | 0.64*** | −22 | 82.91*** | 37 |
| Arginine | | | | | | | | | | |
| L-Citrulline | 42.8*** | 25 | 349.52 | −7 | 559.63** | 4 | 0.69*** | −16 | 77.61 | 28 |
| Dileucine + L- | 45.62*** | 33 | 350.79*** | −7 | 567.19 | 6 | 0.67*** | −18 | 79.14*** | 31 |
| Citrulline | | | | | | | | | | |

Data represented by Mean ± SD (n = 8 in each group). Statistical significances are compared between Exercise Control (G1) versus other treatment groups
(*P Value < 0.05);
(**P Value < 0.01);
(***P Value < 0.001)

### Effect of Supplementation on Immune Health Biomarkers

The synergistic combination of paraxanthine+dileucine significantly increased TH17, Tfh, and significantly decreased TH1, iTreg over control after 28 days of supplementation and exercise. The immune health biomarkers measurements of T helper subsets are shown in table 8.

TABLE 8
Effect on nitric oxide levels. Data are the mean ± SD for n = 8 mice in each group.

| | | Change | | Change | | Change | | Change | | Change |
|---|---|---|---|---|---|---|---|---|---|---|
| | | over | | over | | over | | over | | over |
| | | control | | control | | control | Tfh(%) | control | iTreg(%) | control |
| Treatment | TH1(%) | [%] | TH2(%) | [%] | TH17(%) | [%] | (mg/L) | [%] | (mg/L) | [%] |
| Exercise Control | 93.54 | | 11.46 | | 3.65 | | 7.82 | | 54.47 | |
| Dileucine | 58.86*** | −37 | 53.68*** | 368 | 29.62*** | 712 | 55.72 | 613 | 18.68*** | −66 |
| Paraxanthine | 65.68*** | −30 | 53.63*** | 368 | 31.33*** | 758 | 54.96*** | 603 | 23.68*** | −57 |
| Dileucine + | 18.26*** | −80 | 61.54*** | 437 | 36.01*** | 887 | 78.38*** | 902 | 9.7*** | −82 |
| Paraxanthine | | | | | | | | | | |
| L-Arginine | 53.66*** | −43 | 40.44*** | 252 | 26.16*** | 617 | 44.16*** | 465 | 35.86*** | −34 |
| Dileucine + L- | 13.19*** | −86 | 57.72*** | 404 | 29.71*** | 714 | 66.3*** | 748 | 22.09*** | −59 |
| Arginine | | | | | | | | | | |
| L-Citrulline | 59.71*** | −36 | 38.23*** | 234 | 25.89*** | 609 | 40.87*** | 423 | 38.83*** | −29 |
| Dileucine + L- | 23.23*** | −75 | 55.88*** | 388 | 28.75*** | 688 | 59.26*** | 658 | 24.47*** | −55 |
| Citrulline | | | | | | | | | | |

Data represented by Mean ± SD (n = 8 in each group). Statistical significances are compared between Exercise Control (G1) versus other treatment groups
(*P Value < 0.05);
(**P Value < 0.01);
(***P Value < 0.001)

While multiple embodiments are disclosed, still other embodiments of the disclosure will become apparent to those skilled in the art from the following detailed description, which shows and describes illustrative embodiments of the disclosed compositions, systems and methods. As will be realized, the disclosed compositions, systems and methods are capable of modifications in various obvious aspects, all without departing from the spirit and scope of the disclosure. Accordingly, the drawings and detailed description are to be regarded as illustrative in nature and not restrictive.

What is claimed is:

1. A composition comprising: paraxanthine and dileucine or L-arginine.

2. The composition of claim 1, wherein paraxanthine is present in an amount of from about 2 mg to about 800 mg.

3. The composition of claim 2, wherein paraxanthine is present in an amount from about 20 mg to about 600 mg.

4. The composition of claim 3, wherein paraxanthine is present in an amount from about 50 mg to about 400 mg.

5. The composition of any of claim 1, wherein dileucine is present in an amount of from about 10 mg to about 5,000 mg.

6. The composition of claim 5, wherein dileucine is present in an amount of from about 25 mg to about 2,500 mg.

7. The composition of any of claim 1, wherein L-arginine is present in an amount of about 500 mg to about 6,000 mg.

8. The composition of claim 7, wherein L-arginine is present in an amount of about 1,000 mg to about 3,000 mg.

9. The composition of claim 7, wherein the L-arginine is in the form of a free form, salt, bonded arginine silicate complex, and/or arginine alpha-ketoglutarate.

10. A method of improving muscle strength, muscle size, and/or muscle function comprising: administering to a subject a composition comprising dileucine in an amount of from about 10 mg to about 5,000 mg; and
a) paraxanthine in an amount of from about 2 mg to about 800 mg; or
b) L-arginine in an amount of from about 500 mg to about 6,000 mg.

11. The method of claim 10, wherein the composition further comprises one or more compounds selected from the list consisting of: isoleucine, leucine, and valine, histidine, lysine, methionine, phenylalanine, threonine, tryptophan, valine, creatine, cysteine, glutamine, glycine, proline, tyrosine, carnitine, beta-alanine, taurine, beta-hydroxy beta-methylbutyrate, Omega-3 fatty acids, Vitamin D, whey protein, and other protein extracts from animal, plant or fermentation sources.

12. A method for increasing athletic performance in a subject, comprising: administering a subject a composition comprising dileucine in an amount of from about 10 mg to about 5,000 mg; and
a) paraxanthine in an amount of from about 2 mg to about 800 mg; or
b) L-arginine in an amount of from about 500 mg to about 6,000 mg.

13. A method for promoting weight loss in a subject, comprising: administering to a subject a composition comprising dileucine in an amount of from about 10 mg to about 5,000 mg; and
a) paraxanthine in an amount of from about 2 mg to about 800 mg; or
b) L-arginine in an amount of from about 500 mg to about 6,000 mg.

14. The method of claim 13, wherein the composition further comprises one or more compounds selected from a list consisting of: caffeine, green tea, capsaicin, garcinia cambogia, yohimbine and bitter orange.

15. The method of claim 13, wherein weight loss is promoted through enhancing lipolysis in the subject and wherein the composition further comprises one or more compounds selected from a list consisting of caffeine, green tea extract, L-carnitine, Garcinia cambogia (hydroxycitric acid), capsaicin, ginseng, taurine, silk peptides, catechols, epigallocatechin gallate (EGCG), catechins, proanthocyanidins and octacosanol.

16. A method of improving cognitive performance in a subject comprising administering the composition comprising dileucine in an amount of from about 10 mg to about 5,000 mg; and
a) paraxanthine in an amount of from about 2 mg to about 800 mg; or
b) L-arginine in an amount of from about 500 mg to about 6,000 mg.

17. The method of claim 16, wherein improved cognitive function is measured by an increase in one or more of: attention, information acquisition, information processing, working memory, short-term memory, long-term memory, anterograde memory, retrograde memory, memory retrieval, discrimination learning, decision-making, inhibitory response control, attentional set-shifting, delayed reinforcement learning, reversal learning, the temporal integration of voluntary behavior, speed of processing, reasoning, problem solving and/or social cognition.

18. A method of treating an inflammatory disease or condition in a subject in need thereof comprising administering to the subject a composition comprising dileucine in an amount of from about 10 mg to about 5,000 mg; and
a) paraxanthine in an amount of from about 2 mg to about 800 mg; or
b) L-arginine in an amount of from about 500 mg to about 6,000 mg.

19. The method of claim 18, wherein the composition further comprises a further anti-inflammatory agent wherein the further anti-inflammatory agent exerts anti-inflammatory effects by: inhibiting prostaglandins, increasing macrophage mediated phagocytosis, suppressing cytokine-driven inflammation, inhibiting cytokines, inhibiting histone deacetylation, inhibiting kinases, stimulating PPAR and/or inhibiting proteases.

20. A method for improving joint health in a subject in need thereof comprising: providing the subject with a composition comprising dileucine in an amount of from about 10 mg to about 5,000 mg; and
a) paraxanthine in an amount of from about 2 mg to about 800 mg; or
b) L-arginine in an amount of from about 500 mg to about 6,000 mg.

21. A method for enhancing immune function in a subject, comprising: providing the subject with a composition comprising dileucine in an amount of from about 10 mg to about 5,000 mg; and
a) paraxanthine in an amount of from about 2 mg to about 800 mg; or
b) L-arginine in an amount of from about 500 mg to about 6,000 mg.

22. The method of claim 21, wherein the composition further comprises one or more compounds selected from the list consisting of: Cyclosporine, tacrolimus, rapamycin, Omega-3 fatty acids, Curcumin, S-adenosylmethionine, Zinc, Green tea extract, Frankincense (Boswellia serrata resin), Capsaicin, Cat's claw (uncaria plants, including Uncaria tomentosa and Uncaria guianensis), Schizonepeta tenuifolia, Pomegranate, Moringa oleifera, Ecklonia cava, Limonene, Sunifiram, Sulforaphane, Angelica gigas, Ascophyllum nodosum, Scutellaria baicalensis, celery seed extract, Sesamin, Feverfew, Taurine, Rosmarinic Acid, Evodia rutaecarpa, Green Tea Catechins, Punicalagins, Artemisia iwayomogi, Pyrroloquinoline quinone, N-Acetylcysteine, King Oyster, Methylsulfonylmethane, alpha-lipoic acid, pine pollen, Sophora flavescens, Ophiopogon japonicus, Stephania tetrandra, Crataegus pinnatifida, grape seed extract, Bladderwrack, Paederia foetida, Benfotiamine, Rubus coreanus, Punicic Acid, Sea Buckthorn, Hibiscus rosasinensis, Phellodendron amurense, Resveratrol, Quercetin, Rooibos, Olive leaf extract, Pterostilbene, Eucommia ulmoides, Diindolylmethane, Anatabine, Serrapeptase, Pelargonidin, watercress, Astaxanthin, Piceatannol, Fish Oil, Glutathione, Orthosiphon stamineus, Aronia melanocarpa, blueberry, Tripterygium wilfordii, Boerhaavia diffusa, Whey Protein, Bromelain, Panax ginseng, Aloe vera, cocoa extract, stinging nettle, garlic, Centella asiatica, Astragalus membranaceus, Dendrobium, Vitamin C, Spirulina, Berberine, Ganoderma lucidum, Vitamin C, Vitamin D, Vitamin E, lutein, leucine, dileucine, trileucine, tetraleucine Pau d'arco, AHCC, rhodiola ashwagandha, shiake, maitake, turkey tail, monolaurin, lysine, Ergothioneine, medium chain triglycerides (MCTs) and butyrate.

23. A method for improving cardiovascular health in a subject, comprising: providing the subject with a composition comprising dileucine in an amount of from about 10 mg to about 5,000 mg; and
a) paraxanthine in an amount of from about 2 mg to about 800 mg; or
b) L-arginine in an amount of from about 500 mg to about 6,000 mg.

24. A method for improving blood flow in a subject, comprising: providing the subject with a composition comprising dileucine in an amount of from about 10 mg to about 5,000 mg; and
a) paraxanthine in an amount of from about 2 mg to about 800 mg; or
b) L-arginine in an amount of from about 500 mg to about 6,000 mg.

25. A method for increasing athletic endurance in a subject, comprising: administering to a subject an effective amount of a composition comprising dileucine in an amount of from about 10 mg to about 5,000 mg; and
a) paraxanthine in an amount of from about 2 mg to about 800 mg; or
b) L-arginine in an amount of from about 500 mg to about 6,000 mg.
